(19) United States
(12) Patent Application Publication
(10) Pub. No.: US 2026/0038630 A1
(43) Pub. Date: Feb. 5, 2026

(54) Computational Method for Identifying Biophysical Interactions That Determine Human T-Cell Specificity

(71) Applicants: Jason George, Houston, TX (US); Ailun Wang, Long Island City, NY (US); Xingcheng Lin, Raleigh, NC (US); Herbert Levine, Brookline, MA (US)

(72) Inventors: Jason George, Houston, TX (US); Ailun Wang, Long Island City, NY (US); Xingcheng Lin, Raleigh, NC (US); Herbert Levine, Brookline, MA (US)

(73) Assignee: The Texas A&M University System, College Station, TX (US)

(21) Appl. No.: 19/288,818

(22) Filed: Aug. 1, 2025

Related U.S. Application Data
(60) Provisional application No. 63/678,438, filed on Aug. 1, 2024.

(51) Int. Cl. G16B 15/30; G16B 15/20; G16B 40/20

(52) U.S. Cl. CPC G16B 15/30; G16B 15/20; G16B 40/20

(57) ABSTRACT

Provided herein are computer-implemented methods for training a computational model to predict T cell-antigen specificity and to predict T cell-antigen. Also provided is a system configured to predict T cell-antigen specificity via the computational model tangibly stored on an electronic device. The computational model is trained with a data training set, such as a sparse data training set, that is used as input.

### CROSS-REFERENCE TO RELATED APPLICATIONS

This non-provisional patent application claims benefit of priority under 35 U.S.C. § 119(e) of provisional patent application U.S. Ser. No. 63/678,438, filed Aug. 1, 2024, the entirety of which is hereby incorporated by reference.

### STATEMENT REGARDING FEDERALLY SPONSORED RESEARCH

This invention was made with government support under Grant Number PHY-2019745 awarded by the National Science Foundation. The government has certain rights in the invention.

### INCORPORATION BY REFERENCE OF SEQUENCE LISTING

The Sequence Listing XML file, entitled D8047SEQ.xml, was created on Aug. 1, 2025, and has a size of 53000 bytes. This Sequence Listing is hereby incorporated by reference in its entirety.

### BACKGROUND OF THE INVENTION

### Field of the Invention

The present invention relates generally to the fields of computational modeling and immunology. More specifically, the present invention relates to a computational model for antigen specificity prediction for individual patients.

### Description of the Related Art

The ability of T cell receptors (TCRs) to selectively recognize short antigenic peptides bound to major histocompatibility complex (MHC) molecules underpins a broad spectrum of adaptive immune responses in contexts such as infection, cancer, and autoimmunity. Accurate prediction of peptide recognition by diverse TCR repertoires remains a central challenge in computational immunology, with relevance for vaccine design, immune monitoring, and the development of targeted immunotherapies. Human TCR repertoires are remarkably diverse each comprising an estimated 10⁸ unique clonotypes drawn from a theoretical space of up to 10¹⁹ and also capable of self-non-self discrimination within an extensive (˜10¹³) peptide landscape. This vast combinatorial complexity therefore necessitates the development of robust computational frameworks to complement experimental strategies aimed at understanding repertoire-level TCR specificity.

The number of allowable T cells (10¹⁹) and antigens (10¹³) are staggering, effectively precluding a comprehensive experimental characterization of T cell-antigen specificity However, recent significant advancements have been made in understanding TCR recognition, driven by both the emergence of high-throughput experimental datasets (1,2) and concurrent development of advanced computational frameworks that accurately capture TCR-pMHC interactions (3-13). Among these computational approaches, structurally informed biophysical models of the TCR-peptide interaction (RACER-m) have emerged as particularly effective frameworks to understand TCR specificity (12,13). While these models have demonstrated reasonable accuracy in predicting specificity for a variety of publicly available datasets, their capacity to accurately predict antigen specificity for previously unseen TCR sequences remains unstudied.

Thus, the prior art is deficient in technology that reliably predicts which T cells recognize which antigens. Particularly, the prior art is deficient in computational models that characterize human T-cell antigen specificities by reliably discerning meaningful T-cell antigen pairs from those that do not form good recognition pairs. The present invention fulfills this long-standing need and desire in the art.

### SUMMARY OF THE INVENTION

The present invention is directed to a computer-implemented method for training a computational model to predict T cell-antigen specificity. In this method, a data training set of known T cell-antigen interactions is input into the computational model. An optimized description of amino acid interactions is generated from the data set. The predictive ability of the computational model and the optimized description is tested against known T cell-antigen pairs.

The present invention is directed to a related computer-implemented method that further comprises updating the computational model. In this related method, an updated data training set is inputted into the computational model and an updated description of the optimized description of amino acid interactions is generated from the updated data training set. The predictive ability of the computational model and the updated optimized description is tested against known T cell-antigen pairs. These steps are repeated at least once.

The present invention is further directed to a computer-implemented method for predicting T cell specificity against antigens. In this method, a computational model for T cell-antigen specificity prediction is trained using a data set of known T cell-antigen interactions to generate an optimized energy model of amino acid interactions that determine T cell specificity against antigens. The trained computational model and the optimized energy model are used to predict which T cells recognize an unknown antigen or which unknown T cells recognize a given antigen.

The present invention is directed to a related computer-implemented method that further comprises refining the computational model. In this related method, a new data set of known T cell-antigen interactions is incorporated to train an updated computational model to generate an updated optimized energy model of amino acid interactions that determine additional T cell specificity against additional antigens. The updated trained computational model and the updated optimized energy model are used to predict which of the additional T cells recognize an unknown antigen or which unknown T cells recognize a given additional antigen. These steps are repeated at least once.

The present invention is directed further to a system to predict T cell-antigen specificity that comprises a computational model tangibly stored on an electronic device having at least one processor and at least one memory in communication with the processor. The memory tangibly stores instructions that, when executed by the processor, are configured to perform at least the following functions. The computational model is configured to predict T cell-antigen specificity using a data set of known T cell-antigen interactions, to generate an optimized energy model of amino acid interactions that determine T cell specificity against antigens and to predict via the optimized energy model and binding specificity of an antigenic peptide toward a specific T cell receptor based on the respective peptide sequences thereof. The present invention is directed to a related system where the electronic device further comprises at least one network connection.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the RACER-m model architecture with a schematic representation of the training (top row) and testing (bottom row) processes in RACER-m. Sixty-six crystal structures of known strong binders were used as both training set and template structures for the testing processes, which cover several major clusters of TCR repertoires (MART-1, TAX, 1E6, NLV, and FLU) and other clusters with smaller size.

FIGS. 2A-2C are contact maps of crystal structures 3QDG, 3UTT and 10GA. Each contact map was calculated by measuring the proximity Wi,j between each residues of peptide (residue i) and CDR loops (residue j) based on their mutual distance (d) using a smoothed step function: Wi,j=(1−tanh(d−dmax))/2, where dmax=8.5° A. Only Cβ atoms were used for the mutual distance calculation (except for glycine, where the Cα atom was used).

FIG. 3 shows the relationship between structure and sequence similarities of TCR-pMHC complexes

FIGS. 4A-4B are the performance on the ATLAS dataset. FIG. 4A shows the mutual Q calculation results between all crystal structures in training set of RACER-m, which measures the structural similarity between every pair of structures from the training set. The linkage map shows the hierarchical clustering result based on the pairwise mutual Q values. Color blocks next to the linkage map indicates the corresponding cluster of the crystal structure in the row. FIG. 4B shows the predicted binding energies for ATLAS dataset (open circles and closed dots) in comparison with the binding energies for corresponding weak binders (box plots). Each open circle represents the predicted binding energy for a structure in the training set, while each closed dot represents the predicted binding energy for a testing case from ATLAS dataset. Each training or testing case is associated with 1000 decoy weak binders generated by randomizing the peptide sequence and pairing with the TCR in the corresponding training/testing structure. Box plots represent the distribution of the predicted energies of the decoy weak binders with the box representing the lower (Q1) to upper (Q3) quartiles and a horizontal line representing the median. The whiskers extended from the box by 1.5 IQR, where IQR indicates the interquartile range.

FIG. 5 shows the distribution of predicted binding energies of MART-1 and FLU.

FIGS. 6A-6C show the prediction performance on weak binders generated by mismatching peptides with TCRs. FIG. 6A are receiver operating characteristic (ROC) curves for RACER-m classification performance on differentiating weak binders generated by mismatching peptides from NLV, TAX, FLU, and 1E6 clusters with MART-1 TCRs from MART-1 strong binders with the same set of TCRs. FIG. 6B are ROC curves for RACER-m classification performance on distinguishing MART-1 strong binders from mismatched weak binders generated by pairing MART-1-specific peptides with TCRs from NLV, TAX, FLU, and 1E6 clusters. FIG. 6C shows that when TAX A6 TCR is paired with MART-1 peptide ELAGIGILTV (SEQ ID NO: 1), the z score of the mismatched TCR-pMHC pair (triangle) resembles the values from the strong binders (violin shape) formed by the same peptide and TCR RD1-MART1^{High} and its point mutants, which was engineered from A6. In the reverse scenario, TCR RD1-MART1^{High} shows lower z score (cross) than TAX strong binders (violin shape) when paired with TAX specific peptide LLFVYPVYV (SEQ ID NO: 2).

FIGS. 7A-7C validate the predictive power of RACER-m with external datasets. FIG. 7A shows the prediction results of RACER-m on the HLA-A*02:01-restricted systems from 10× Genomics dataset collected from five healthy donors. A total of 1741 unique pairs of TCR-peptide sequences were tested, and the prediction results of z score were grouped by the immunological profile of the test TCR-pMHC pairs and depicted as box plots. FIG. 7B is a comparison of classification performance between RACER-m and NetTCR-2.0 (11) on a curated list of public TCR-pMHC repertoires (12, 42) composed of both strong binders and mismatched weak binder. Because of the restriction of NetTCR-2.0 on the peptide length (9-mer), there are no data from NetTCR-2.0 for the two 10-mer peptides (KLVALGINAV, SEQ ID NO: 3 and ELAGIGILTV, SEQ ID NO: 1). FIG. 7C shows the classification performance of RACER-m on another set of TCR-pMHC test TCR-pMHC pairs (34), AUROC, area under the ROC curve.

FIGS. 8A-8B show the distribution of optimal sequence similarity score (FIG. 8A) and the distribution of RACER-m predicted z-scores (FIG. 8B) for strong binders in VDJd.

FIGS. 9A-9C show RACER-m's performance on differentiating strong binders from point-mutant weak binders. FIG. 9A is a distribution of z scores from strong binders of 1E6 TCR and weak binders from point mutagenic screen. FIG. 9B is a ROC curve for RACER-m classification performance using the strong and point-mutant weak binders for 1E6 TCR. FIG. 9C is a comparison of RACER-m and NetTCR-2.0 in classification of strong and point-mutant weak binders from ATLAS dataset. Here, RACER-m predictions used the known crystal structure selected by the sequence similarity calculation results as a representative template for threading each test case.

FIGS. 10A-10C are schematic representations of the workflow. FIG. 10A shows the experimental setup. Patient-derived and healthy donor peripheral blood mononuclear cell (PBMC) or bone marrow (BM) samples are labeled with sample-specific hashtags, pooled, and stained with a DNA-barcoded tetramer library, where each tetramer is uniquely encoded by a specific DNA barcode. Tetramer-binding (PE and/or APC) cells are sorted and sequenced to determine both tetramer identity and the paired TCRa/P sequences. FIGS. 10B-10C show the training and testing pipeline. The training phase utilizes crystal structures and synthetic in silico data, for example, but not limited to AlphaFold-derived, Chai-1-derived, or Boltz-1 derived TCR-pMHC complexes, to build or refine the predictive model. The testing phase classifies unknown TCRs, predicting their recognition of one of several candidate peptides such as MART-1, FLU, CMV-NLV).

FIGS. 11A-11B are the Z-score binding criteria. In FIG. 11A binding confidence is determined by Z-score relative to the distribution of known binders. Confident (dark gray), intermediate confidence (gray), and low confidence (light gray) is assigned to determine binding and non-binding TCR-peptide pairs. In FIG. 11B shows TCR selection criteria for determining binding/non-binding is determined by identifying the peptide exhibiting maximal z-score.

FIGS. 12A-12D show the distribution of Z-scores for MART-1 (FIG. 12A), FLU (FIG. 12B), and CMV-NLV (FIG. 12C) in Training Set 1, which includes the ATLAS and MDACC_1. The MART-1 and CMV-NLV distributions exhibit two distinct peaks, indicating the presence of two different structural conformations, whereas the FLU distribution shows a single peak. These Z-score distributions were obtained after refining the structural template library using mutual Q similarity metrics (FIG. 12D) to select the optimal templates for binding energy calculations.

FIG. 13 shows Receiver Operating Characteristic (ROC) curve results for test set 1, using the mutual Q method as the criterion for clustering TCRs specific to MART-1, FLU, and CMV-NLV.

FIGS. 14A-14E shows TCRdist-based analysis for MDACC_3 and MDACC_4. FIGS. 14A and 14C are ROC curves demonstrating model performance in distinguishing binder TCRs for MDACC_3 and MDACC_4, respectively. FIGS. 14B and 14D show the corresponding TCRdist-based clustering results, illustrating the sequence-based grouping of TCRs. FIG. 14E shows the TCR clustering of the ATLAS training set using TCRdist, highlighting sequence-based distances between experimentally identified crystal structure TCRs specific for MART-1, FLU, and NLV epitopes.

FIG. 15A shows the structure generated by AlphaFold, Chai-1 and Boltz-1. The CDR3α/β, peptide, and MHC (colored regions) represent sequence information derived from the MDACC datasets, whereas the variable regions (Va and Vp), excluding CDR3α/β, are based on sequence information from the best template selected by RACER-m for each respective MDACC case. FIGS. 15B-15C show the structure generated by Chai-1 and Boltz-1.

FIGS. 16A-16B shows the comparison of the refined fifteen-case selection approach. FIG. 16A shows the TCR clustering of the MDACC_5 dataset, where gray nodes represent all TCRs, and pink nodes indicate the selected cases used for in silico structure generation and incorporation into the training set. FIG. 16B is an ROC curve showing the performance of the selected cases compared to the thirty-case random selection.

FIGS. 17A-17B are ROC curves (FIG. 17A) and clustering results (FIG. 17B) for test set 4. These include in silico derived synthetic 3D structures generated with AlphaFold3 to address the diverse TCR clones for MART-1. The curves illustrate the results for different random subsets excluded from the test set and added to the training set, with their structures generated using AlphaFold3. The average ROC curve is presented alongside the confidence interval (CI) to indicate variability and reliability across random trials. The computed ROC-AUC values for each peptide were reported with 90% confidence intervals, providing a robust assessment of the model's reliability and predictive power.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the terms “sparse” and “sparse sampling” are interchangeable and refers to situations involving an analysis where the systems in question represent a small subset of the allowable diversity permitted by the physical system.

In one embodiment of the present invention, there is provided a computer-implemented method for training a computational model to predict T cell-antigen specificity, comprising a) inputting into the computational model a data training set of known T cell-antigen interactions; b) generating an optimized description of amino acid interactions from the sparse data set; and c) testing the predictive ability of the computational model and the optimized description against known T cell-antigen pairs.

Further to this embodiment the computer-implemented method comprises updating the computational model via the steps of d) inputting an updated data training set into the computational model; and e) generating an updated description of the optimized description of amino acid interactions from the updated data training set; and f) testing the predictive ability of the updated computational model and the updated optimized description against known T cell-antigen pairs. In another further embodiment, the computer-implemented method comprises repeating at least once steps d), e) and f).

In all embodiments, the data training set may comprise a sparse sampling of the known T cell-antigen interactions. In an aspect thereof, the sparse sampling of the known T cell-antigen interactions may comprise a plurality of human MHC-I allele variant peptide sequences and protein crystal structures thereof. Particularly, the human MHC-I allele variant is HLA-A*02:01. Also in all embodiments, the optimized description of amino acid interactions may be a combined sequence-structural model of T cell receptor-pMHC specificity that comprises biophysical information.

In another embodiment of the present invention, there is provided computer implemented method for predicting T cell specificity against antigens, comprising a) training a computational model for T cell-antigen specificity prediction using a data set of known T cell-antigen interactions to generate an optimized energy model of amino acid interactions that determine T cell specificity against antigens; and b) using the trained computational model and the optimized energy model to predict which T cells recognize an unknown antigen or which unknown T cells recognize a given antigen.

Further to this embodiment, the computer-implemented method may comprise refining the computational model via the steps of c) incorporating a new data set of known T cell-antigen interactions to train an updated computational model to generate an updated optimized energy model of amino acid interactions that determine additional T cell specificity against additional antigens; and d) using the updated trained computational model and the updated optimized energy model to predict which of the additional T cells recognize an unknown antigen or which unknown T cells recognize a given additional antigen. In another further embodiment, the computer-implemented method comprises repeating at least once steps c) and d).

In all embodiments, the data training set may be a sparse data set of the known T cell-antigen interactions. In an aspect thereof, the known T cell-antigen interactions may comprise a plurality of human MHC-I allele variant peptide sequences and protein crystal structures thereof. Particularly, the human MHC-I allele variant is HLA-A*02:01. Also in all embodiments and aspects thereof, the trained computational model and the optimized energy model may resolve the T cell-antigen specificity of the unknown T cell receptors against tumor antigens and viral antigens. In addition, the optimized energy model may be utilized for predicting binding specificity of an antigenic peptide toward a specific T cell receptor based on the respective peptide sequences thereof.

In yet another embodiment of the present invention, there is provided a system to predict T cell-antigen specificity comprising a computational model tangibly stored on an electronic device having at least one processor and at least one memory in communication with the processor, the memory tangibly storing instructions that, when executed by the processor, are configured to at least: train the computational model to predict T cell-antigen specificity using a data set of known T cell-antigen interactions; to generate an optimized energy model of amino acid interactions that determine T cell specificity against antigens; and to predict via the optimized energy model binding specificity of an antigenic peptide toward a specific T cell receptor based on the respective peptide sequences thereof. Further to this embodiment, the electronic device comprises at least one network connection.

In both embodiments, the data set of the known T cell-antigen interactions may be a sparse data set. In an aspect of both embodiments, the sparse data set may comprise a plurality of human MHC-I allele variant peptide sequences and protein crystal structures thereof. In both embodiments, the computational model may be configured to predict which T cells recognize an unknown antigen or which unknown T cells recognize a given antigen.

Provided herein is a specialized computational modeling framework configured to guide the identification of antigen specific T cell receptors (TCRs). The RACER-m framework is leveraged to distinguish tumor-specific TCRs from those targeting viral epitopes. In a non-limiting example clinical context of allogeneic hematopoietic stem cell transplantation (allo-HSCT) provides one such setting to evaluate and apply this strategy, as patient- and donor-derived expanded TCR repertoires are efficiently obtained and sequenced directly from peripheral blood or bone marrow samples. Alternatively, the computational modeling framework may be configured to identify T cells targeting any antigens, for example, cancer, viral diseases, infectious diseases, and autoimmunity.

Following sequencing, TCR specificity was computationally predicted and subsequently validated experimentally through affinity-driven tetramer sorting, then evaluated iteratively. To address the limitation of sparse TCR training data in a discrete ligand space sequences obtained from HSCT-derived repertoires were incorporated, experimentally validated TCR-pMHC complexes, and additional in silico-derived TCR-pMHC structural models generated using several recent computational approaches, including AlphaFold3 (14). Furthermore, peptide-specific binder distribution profiles were established to more accurately characterize antigen recognition.

The integration of structural features and sequence-based clustering via TCRdist (15,16) significantly enhanced the model's ability to resolve the specificity of previously unseen TCRs against viral and tumor antigens. The specialized approach consistently achieved high predictive accuracy (>80%) across multiple TCR test cohorts. Collectively, this illustrates the predictive power of structurally informed models. This joint experimental and modeling approach also establishes an iterative strategy for continuous model refinement through sequential incorporation of new structural and repertoire data as they become available, which we anticipate will be of broad use in diverse clinical contexts (13, 17-20).

The following examples are given for the purpose of illustrating various embodiments of the invention and are not meant to limit the present invention in any fashion.

### Example 1

### Methods for Training and Testing the RACER-m Model

### RACER-m Model

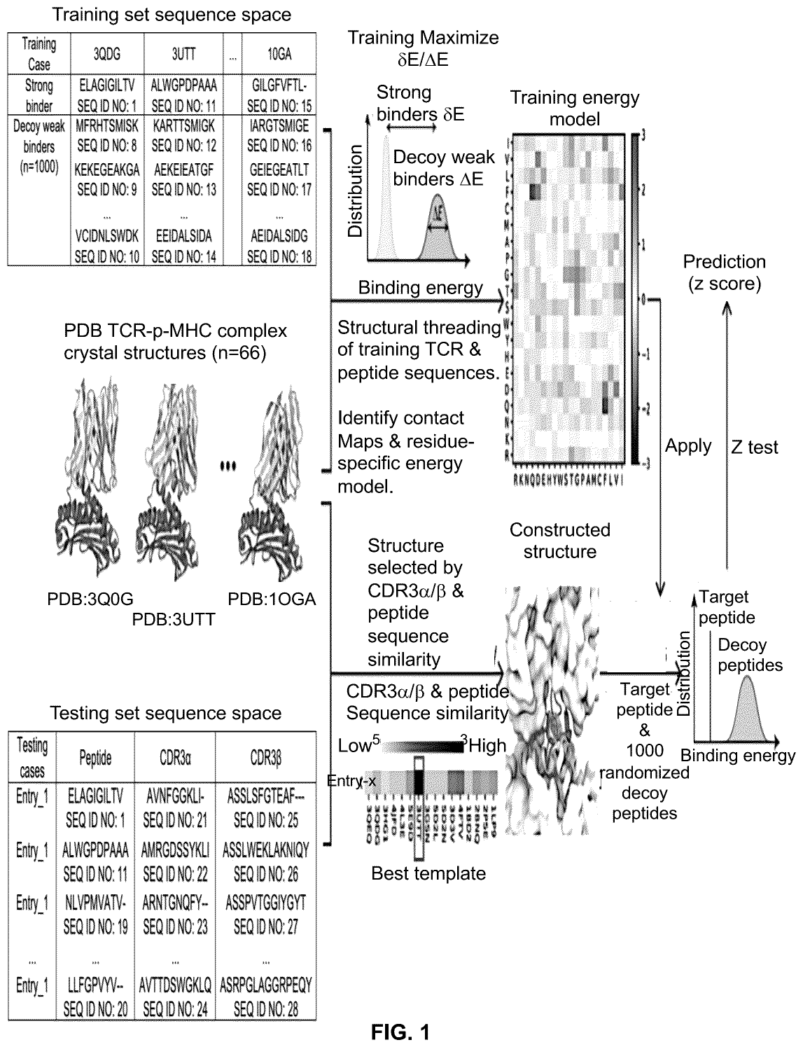

To predict the binding affinity between a given TCR-peptide pair, we used a pairwise energy model was used to assess the TCR-peptide binding energy (14). The CDR3α and CDR3β regions were used to differentiate between different TCRs because CDR3 loops primarily interact with the antigen peptides, while CDR1 and CDR2 interact with MHC (15). However, the binding energy was evaluated on the basis of the entire binding interface between TCR and peptide. As illustrated in FIG. 1, included were 66 experimentally determined TCR-pMHC complex structures and three additional TCR-pMHC complex structures composed of experimentally determined pMHC complexes with corresponding TCR structures as strong binders for training an energy model, which was subsequently used to evaluate binding energies of other TCR-peptide pairs based on their CDR3 and peptide sequences. In addition, for each strong binder, 1000 decoy binders were generated by randomizing the peptide sequence. These 69,000 decoys constitute an ensemble of weak binders within our training set.

To parameterize this energy model, the parameters were optimized by maximizing the gap of binding energies between the strong and weak TCR-peptide binders, represented by δE in FIG. 1. The resulting optimized energy model will be used for predicting the binding specificity of a peptide toward a given TCR based on their sequences. Further details regarding the calculation of binding energy are provided below.

### Detailed Calculation of TCR-Peptide Binding Energies

To evaluate the binding affinity between a TCR and a peptide, RACER-m used the framework of the AWSEM force field (16),which is a residue-resolution protein force field widely used for studying protein folding and binding (16,17). To adapt the AWSEM force field for predicting TCR-peptide binding energy, we used its direct protein-protein interaction component was used to calculate the inter-residue contacting interactions at the TCR-peptide interface. Specifically, the Cβ atoms (except for glycine, where Cα atom was used instead) of each residue were used to calculate the contacting energy using the following expression

$${V}_{d i r e c t} = {\sum }_{i ∈ TCR , j ∈ peptide} {γ}_{i , j} ( {a}_{i} , {a}_{j} ) {Θ}_{i , j}^{I} \qquad Eq . 1$$

In Eq. ⊖i,j represents a switching function that defines the effective range of interactions between each amino acid from the peptide and the TCR

$${θ}_{i , j}^{I} = \frac{1}{4} \{ 1 + tanh [ 5. \times ( {r}_{i , j} - {r}_{min}^{I} ) ] \} \{ 1 + tanh [ 5. \times ( {r}_{max}^{I} - {r}_{i , j} ) ] \} \qquad Eq . 2$$

$$where$$

$${r}_{min}^{I} = 6.5 Å$$

$$and$$

$${r}_{max}^{I} = 8.5 Å .$$

The coefficients γ_{i,j}(aᵢ, aⱼ) define the strength of interactions based on the types of amino acids (aᵢ, aⱼ). The γ_{i,j}(aᵢ, aⱼ) coefficients are also the parameters that are trained in the optimization protocols described as follows.

### Optimization of Energy Model for Predicting the TCR-Peptide Binding Specificity

To predict the binding specificity between a given TCR and peptide, the energy model is trained using interactions gathered from the known strong binders and their corresponding randomly generated decoy binders. Following a previous protocol (14), the energy model of RACER-m was trained to maximize the gap between the binding energies of strong and weak binders. In addition, a larger training set was used to achieve a more comprehensive coverage of the structural and sequence space. Specifically, the binding energies were calculated from individual strong binders (E_{strong}) and their corresponding decoy weak binders (E_{decoy}) as described in Eq. 1. Then the average binding energy of the strong ( [symbol] E_{strong} [symbol] ), the average binding energy of the decoy weak binders ( [symbol] E_{decoy} [symbol] ), and the SD of the energies of the decoy weak binders (ΔE) were calculated.

To train the model, the parameters γ_{i,j}(aᵢ, aᵢ) were optimized to maximize δE/ΔE, where δE= [symbol] E_{decoy} [symbol] − [symbol] E_{strong}, resulting in the maximal separation between strong and weak binders. Mathematically, δE can be represented as A^{T}γ, where

$$A = 〈 {ϕ}_{decoy} 〉 - 〈 {ϕ}_{strong} 〉 \qquad Eq . 3$$

Furthermore, the SD of the decoy binding energies ΔE can be calculated as ΔE²=γ^{T}Bγ, where

$$B = 〈 {ϕ}_{decoy} {ϕ}_{decoy}^{T} 〉 - 〈 {ϕ}_{decoy} 〉 {〈 {ϕ}_{decoy} 〉}^{T} \qquad Eq . 4$$

here, ϕ takes the functional form of V_{direct} and summarizes interactions between different types of amino acids. Therefore, the vector A specifies the difference in interaction strengths for each pair of amino acid types between the strong and decoy binders, with a dimension of (1, 210), while the matrix B is a covariance matrix with a dimension of (210, 210).

With the definition above, maximizing the objective function of δE/ΔE can be reformulated as maximization of A^{T}γ/\sqrt{γ^{T}Bγ}. This maximization can be effectively achieved through maximizing the functional objective R(γ)=A^{T}γ−λ₁\sqrt{γTBγ}. By setting ∂R(γ)/∂γ^{T} to 0, the optimization process leads to γ∝B⁻¹A, where γ is a (210, 1) vector encoding the trained strength of each type of amino acid-amino acid interactions. For visualization purposes, the vector γ is reshaped into a symmetric 20-by-20 matrix, as shown in FIG. 1. In addition, a filter is applied to reduce the noise caused by the finite sampling of decoy binders. In this filter, the first 50 eigenvalues of the B matrix are retained, and the remaining eigenvalues are replaced with the 50th eigenvalue.

Construction of Target TCR-pMHC Complex Structures from Sequences

Because RACER-m calculates the binding energy based on the interaction contacts between a given peptide and a TCR, it relies on the 3D structure of the TCR-pMHC complex for contact calculation. Although the training data include a 3D structure for each of the TCR-peptide strong binders, we usually lack 3D structures for most of the testing cases. To address this limitation, we used the software MODELLER (18) to construct a structure based on the target peptide/CDR3 sequences in the test TCR-pMHC pair and a template crystal structure selected from the training set.

Specifically, for each testing TCR-pMHC pair, a position-wise uniform Hamming distance was computed between the target sequence and each of the sequences from the 66 training strong binders with complete TCR-pMHC complex structures, separately for peptide, CDR3α, and CDR3β regions. Then, sequence similarity scores were assigned to peptide, CDR3α, and CDR3β, respectively, with the number of amino acids that remain the same between target and template sequences. To calculate a composite similarity score for the target TCR-peptide complex, we summed the similarity scores of the CDR3α and CDR3β regions and multiplied this sum by the peptide similarity score. The template structure with the highest similarity score was selected as the template for the subsequent sequence replacement using MODELLER (FIG. 1).

To perform the sequence replacement, the peptide, CDR3α, and CDR3β sequences in the template structure were replaced with the corresponding target sequences in the testing TCR-peptide pair. The location of the target sequence in the template structure was determined by aligning the first amino acid of the target sequence with the original template sequence. If the two sequences had different lengths, then the remaining locations were patched with gaps. This sequence alignment and the selected template structure were then used as input for MODELLER to generate a new structure. The constructed structure was then used for the estimation of the binding energy of the testing TCR-pMHC pair.

### Generation of Weak Binders by Mismatchinq Sequences of Known TCR-Peptide Pairs

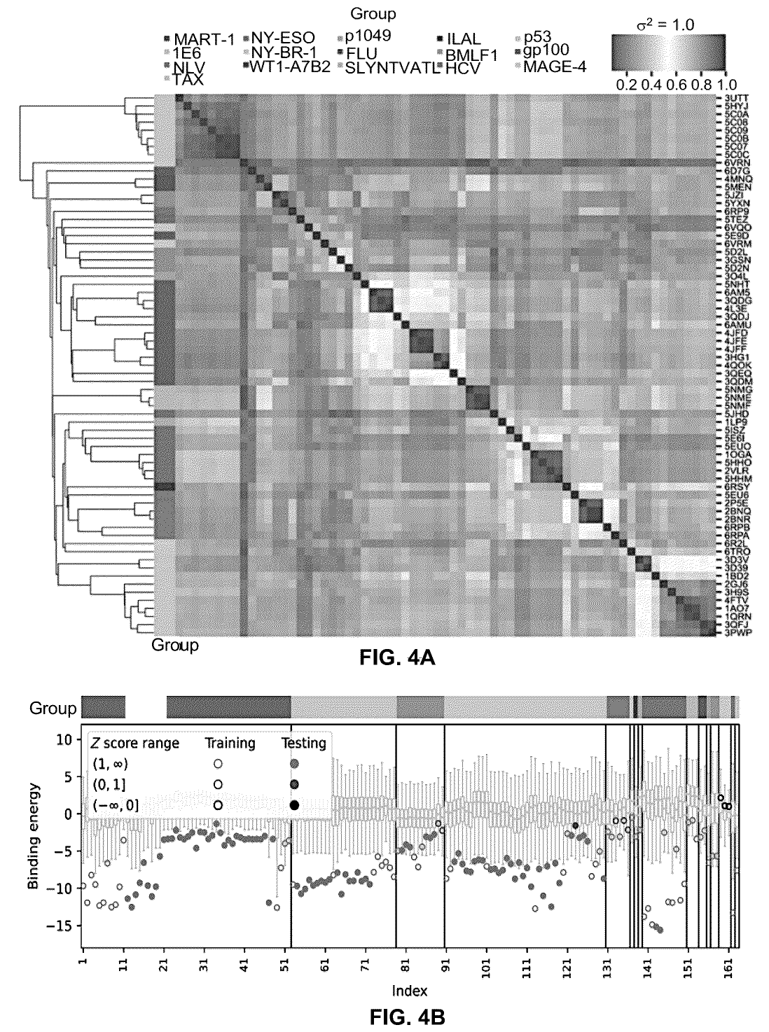

To test the performance of RACER-m in distinguishing strongly bound TCR-peptide pairs from weak binders, a set of weak binders was generated by introducing sequence mismatches between the peptides and TCRs from the known strongly bound TCR-peptide pairs. As shown in FIGS. 4A-4B the strong binders were grouped on the basis of their immunological systems, such as MART-1 and TAX. Note that pairs within the same group also share similar TCR-peptide structural interfaces.

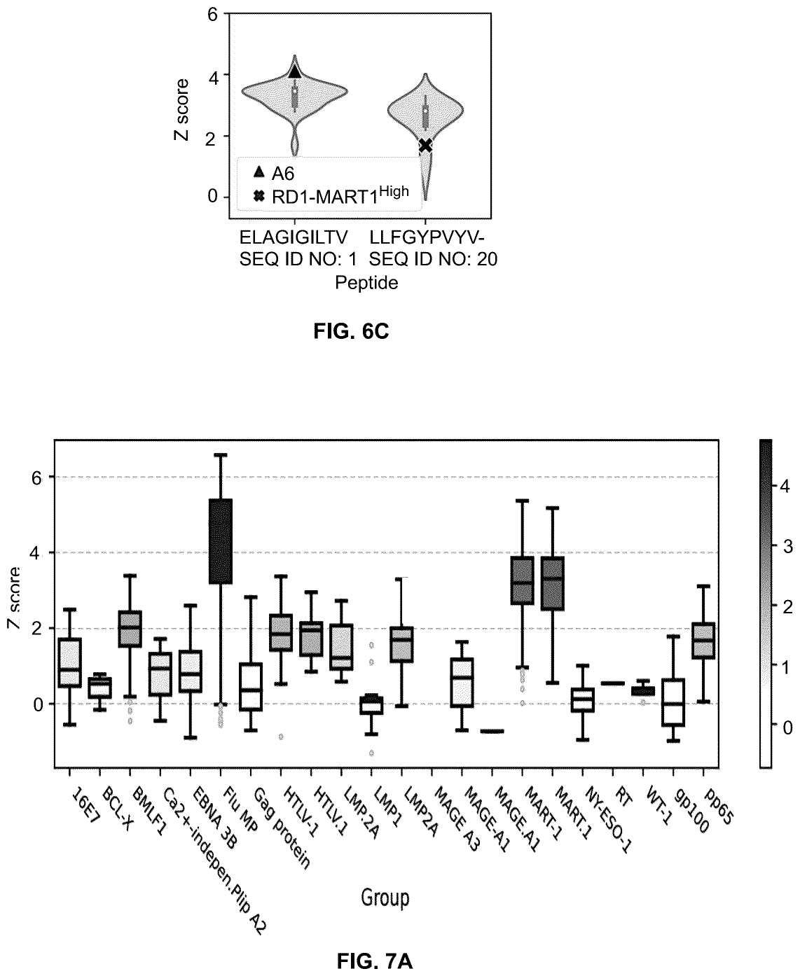

To generate the weak binders, we mismatched the sequences of peptides and the CDR3α/β pairs from different groups. For example, 36 pairs of MART-1-specific CDR3α/β sequences were mismatched with seven non-MART-1 peptides to form weak binders for FIG. 6A while five MART-1-specific peptides were mismatched with 35 pairs of non-MART-1 CDR3α/β sequences to form weak binders in FIG. 6B. The newly generated combinations of sequences were then used to create 3D structures of the TCR-pMHC complexes, following the protocol specified in the “Construction of target TCR-pMHC complex structures from sequences” section.

### Mutual Q Calculation

To quantify the structural distances between the 66 crystal structures of TCR-pMHC complexes, a pairwise mutual Q score was used to calculate the structural similarity between every pair of the 66 structures. Because our focus is on the contact interface between the peptide and the CDR3α/CDR3ρ loops of the TCR, the mutual Q score was computed between these regions. A similar protocol used in (19) was adopted and calculated the mutual Q score between structures A and B with the following expression

$${Q}^{A , B} = c {\sum }_{i ∈ peptide , j ∈ CDR 3} exp [ - \frac{{( {r}_{ij}^{A} - {r}_{ij}^{B} )}^{2}}{2 {σ}^{2}} ] \qquad Eq . 5$$

where l and j are indices of atoms from the peptide and CDR3 loops, respectively. And denote the contact distances between atom l and j in structure A and B, respectively. For simplicity, σ was set as 1 Å instead of using the sequence distance between l and j as done in (19). The coefficient c normalizes the value of Q to fall within the range of 0 and 1. This definition ensures that a larger value of Q indicates a greater structural similarity between the two TCR-pMHC pairs.
Prediction Protocols with NetCR-2.0

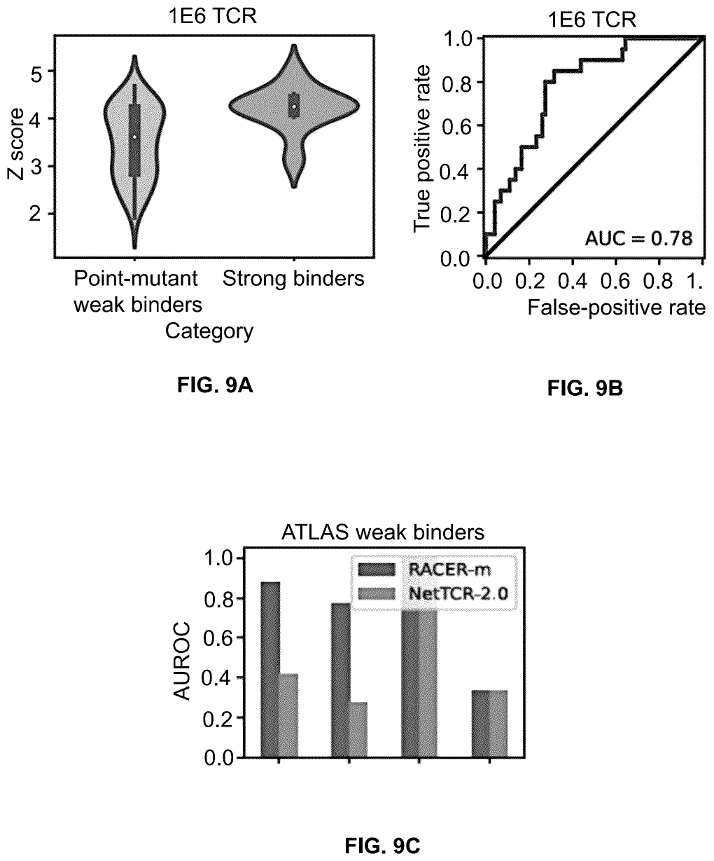

To test the predictive performance of RACER-m, we compared the prediction accuracy of RACER-m with NetTCR-2.0, another widely used computational tool trained with a convolutional neural network model, as described by Montemurro et al. (20). To ensure a fair comparison, the NetTCR-2.0 model was retrained with the dataset with paired α/ρ TCR CDR3 regions and a 95% partitioning threshold (file train_ab_95_alphabeta.csv, provided in github.com/mnielLab/NetTCR-2.0. The trained model was then used to classify the strong and weak binders, as shown in FIG. 9C. Because of the peptide length restriction in the application of NetTCR-2.0, peptides longer than nine residues were excluded from the testing prediction.

### Results

Model Development and Identification of TCR-Peptide Pairs with Structural Templates

Model development built on a previous RACER framework developed primarily on the mouse MHC-II I-E^{k} system (14). The RACER multi-template (RACER-m) approach, represents a comprehensive pipeline that leverages published crystal structures of known human TCR-pMHC pairs.

All 66 HLA-A*02:01-restricted systems with a TCR-pMHC published structure [Protein Data Bank (PDB)/Immune Epitope Database (IEDB)] available through rcsb.org were used as the structures of strong binders for training (21, 22). Their 66 corresponding peptide and TCR variable CDR3a and CDR33 sequences were also used, and this list of TCR-pMHC pairs was further augmented by identification of all reported TCR-pMHC pairs in the publications that referenced the above structures, as part of the “ATLAS dataset.” In addition, the ATLAS database containing affinity information (K_{d}) for related TCR-peptide pairs (23) was used for cases where either a TCR or a epitope had substantial overlap with that of the sequences having structures. A threshold of 200 nM was used to define strong binders to be included in the ATLAS dataset, based on the reported K_{d}. Last, grouping by template was performed using hierarchical clustering based on structural similarity using an approach previously developed in the protein folding community (19,24) followed by hierarchical clustering. In total, 163 unique TCR-peptide pairs and 66 structural templates were identified for training and validation.

Next the structural diversity of training templates was assessed by pairwise evaluation of structural similarity using a previously developed method referred to as mutual Q (21, 25). Mutual Q similarity defines a structural distance metric consisting of a sum of transformed pairwise distances between each residue in two structures normalized within the range of 0 to 1, which was then used to perform hierarchical clustering. It was found that the identified structural clusters largely partition TCR-pMHC systems according to immunological function (for example, systems sharing a conserved antigen) with a few exceptions (FIG. 4A). Despite our focus only on a specified HLA-restricted repertoire, the analysis nonetheless revealed significant clustering heterogeneity across all included systems: In some cases (e.g. MART-1, TAX), substantial heterogeneity was observed and associated with significant pairwise dissimilarity of TCR and peptide sequences. This, together with cross-cluster structural diversity, is a consequence of global sparsity given limited observed structures. On the other hand, we also identified structurally homogeneous clusters comprised of TCR-pMHC systems possessing near-identical pairwise sequence similarity (e.g. 1E6), yet these systems have substantial differences in binding affinity, consistent with earlier predictions (26, 27). This simultaneous manifestation of global sparsity and local resolvability amongst TCR-peptidesystems with identical HLA restriction represents a dual challenge for the developmentof robust predictive models of TCR-peptide specificity.

Given the inter-cluster structural diversity for TCR-pMHC complexes as well as the intra-cluster variability, it is necessary to suitably select a list of structures with sufficient coverage of the identified structural clusters as training data for the model and structural templates for test cases. In particular, it was hypothesized that the hybrid structural and sequence-based methodology could benefit from the inclusion of multiple template structures, and the modeling approach presented here was developed with this motivation in mind.

The flow chart in FIG. 1 illustrates the training (top row) and testing (bottom row) algorithm in RACER-m. For training, contact interactions between peptide and TCR were calculated for each of the strong binding pairs with available TCR-pMHC crystal structures. Here, contact interactions were defined by a switching function based on the distance between structural residues and a characteristic interaction length (see Methods). For each strong binder, 1000 decoy (weak binder) sequences were generated by pairing the original TCR with a randomized version of the peptide. Contact interactions derived from the topology of known TCR-pMHC structures, together with a pairwise 20-by-20 symmetric amino acid energy matrix, determine total binding energy. Each value of the energy matrix corresponds to a particular contribution by an amino acid combination, with negative numbers corresponding to attractive contacts. The training objective aims to select the energy matrix that maximizes separability between the binding energy distributions of strong and weak binders.

In the testing phase, a sequence threading methods is used to construct 3D structures for testing cases that lack a solved crystal structure. Here, constructed structures are based on using a chosen known template with shortest (CDR3α/β and peptide) sequence distance to the specific testing case. Using the constructed 3D structure, a contact interface can be similarly calculated for each testing case, and 1000 decoy weak binders can be generated by randomizing the peptide sequence. The optimal energy model is then applied to assign energies to the testing TCR-pMHC pair and decoy binders, and the testing pair is identified as a strong binder if its predicted binding energy is substantially lower than the decoy energy distributions based on a standardized z score. Here, z score calculation was adopted from the statistical z test applied to the predicted binding energy of test TCR-pMHC pairs and decoy weak binders, the latter of which were used as a null distribution to compare against a given test binder. The z score of binding energies is defined as σ, where is the average predicted binding energy of decoy weak binders, Eₜₑₛₜ is the predicted binding energy of the testing TCR-pMHC pair, and σ_{decoy} is the standard deviation (SD) of the binding energies of decoy weak binders. While model output is composed of continuous values of energy (or normalized z score), we consider test TCR-pMHC pairs with z scores exceeding 1 to be strong binding for categorization purposes.

Structural Information Enhances Recognition Specificity of pMHC-TCR Complexes

RACER-m was developed to explicitly leverage the available structural information obtained from experimentally determined TCR-pMHC complexes for test predictions. While a prior modeling effort (14) relied on a single structural template for both training and testing and achieved reasonable results given reduced training data, structural differences became prominent as the testing data expanded to include additional TCR and peptide diversity, which resulted in reduced predictive utility. Structural variation has been previously observed and quantified in high molecular detail (21, 28) using docking angles (27) and interface parameters.

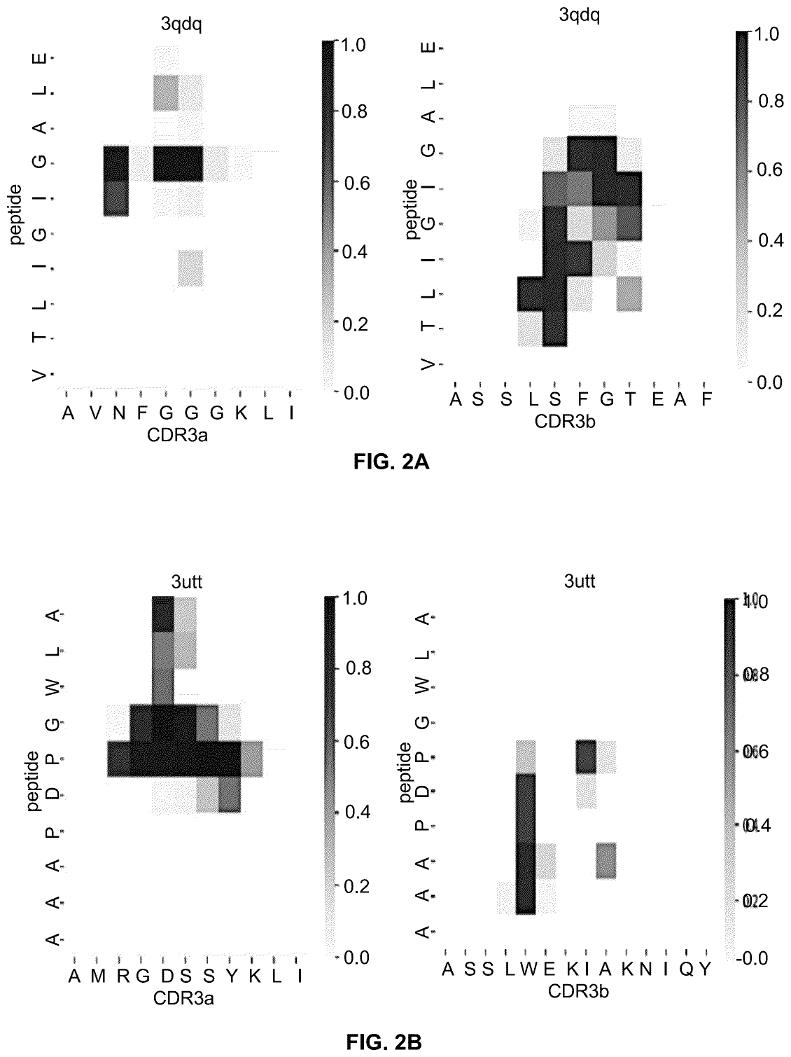

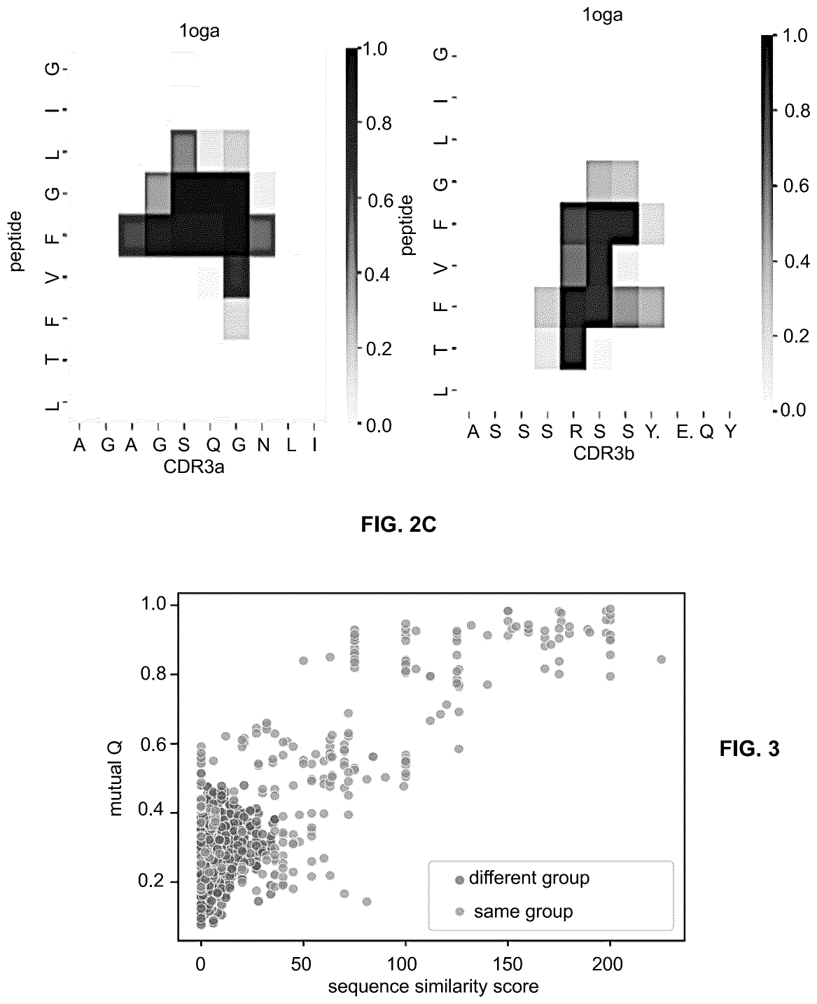

For HLA-A*02:01 TCR-pMHC systems, the docking angles (between the peptide binding groove on the MHC and the vector between the TCR domains, which corresponds to the twist of the TCR over the pMHC) ranged from 29⁰ to 73.1°, while the incident angle varied from 0.3° to 39.5° (21,28, 30). The observed structural differences among different TCR-pMHC complexes suggest that a single TCR-pMHC complex structure may not accurately represent the contact interfaces of other TCR-pMHC complexes, particularly those with substantially different docking orientations. These distinct docking orientations lead to large variations in the contact interfaces between peptide and CDR3α/β loops, which can be observed from the diversity in contact maps as shown in FIGS. 2A-2C. RACER-m overcomes this limitation by the inclusion of 66 TCR-pMHC crystal structures, which are distributed over distinct structural groups, including MART-1, 1E6, TAX, native Cytomegalovirus (NLV), and influenza (FLU) and serve as both the training dataset and reference template structures fortesting cases.

In testing TCR-peptide pairs, all corresponding crystal structures were omitted from predictions. Thus, selecting an appropriate template from available structures became crucial for accurately reconstructing the TCR-pMHC interface and estimating the binding energy. To accomplish this, RACER-m assumed that high sequence similarity corresponds to high similarities in the structure space, which is supported by the correlation between mutual Q score and sequence similarity measured from the 66 solved crystal structures of TCR-pMHC complexes (FIG. 3). This assumption was implemented by calculating sequence similarity scores of the testing peptide and TCR CDR3α/β sequences with those of all 66 reference templates. In each case, a position-wise uniform hamming distance on amino acid sequences was calculated to quantify the similarity. The sum of CDR3α and CDR3β similarities generated the TCR similarity score, and a composite score was created by taking the product of peptide and TCR scores (see Methods). The template structure having the highest sequence similarity was then selected as the template for threading the sequences of the testing TCR-peptide pair.

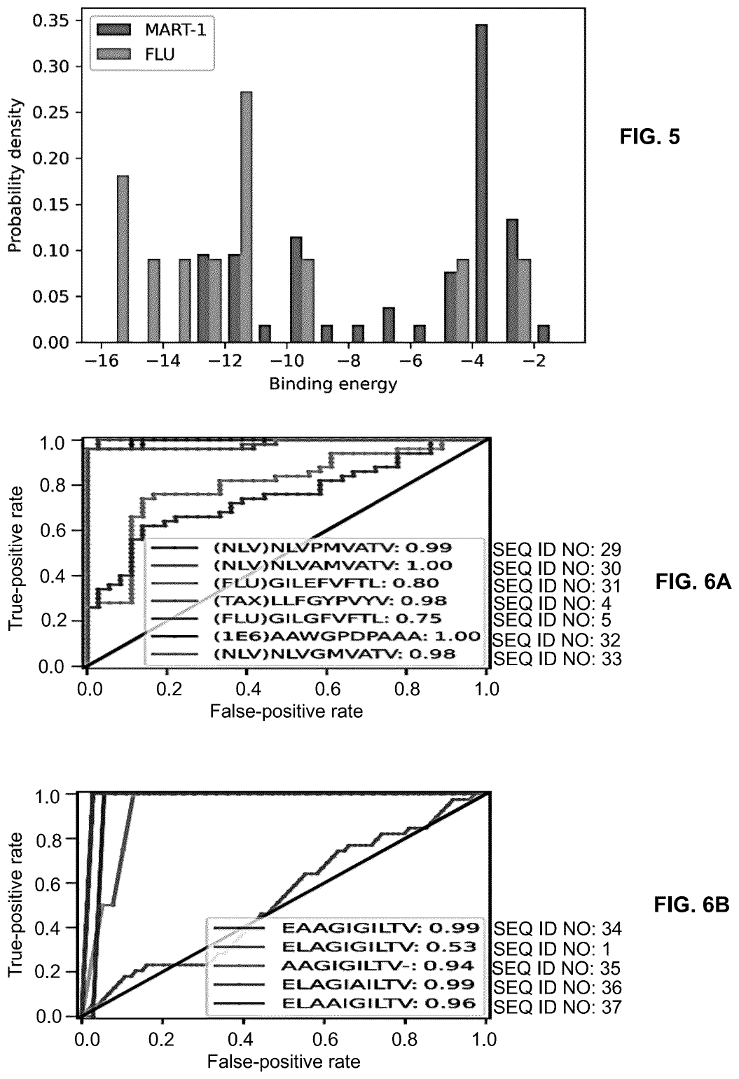

To evaluate the extent to which the RACER-m approach can address global sparsity by accurately recapitulating observed specificity in the setting of limited training data, we trained a model using 42.3% of the total experimentally confirmed strong binders (in addition to the 66 HLA-A*02:01 TCR-pMHC crystal structures plus structures with PDB ID 3GSR, 3GSU, and 3GSV for NLV peptide strong binders (30)) which sparsely cover all the structural groups involved in the mutual Q analysis shown in FIG. 4A. The remaining 57.7% of TCR-peptide sequences that lack solved structures were used as testing cases to validate the sensitivity of the trained energy model. RACER-m effectively recognizes strong binding peptide-TCR pairs and correctly predicts 98.9% of the testing TCR-pMHC pairs using the criteria that z score is greater than 1. Among the 94 testing pairs, only one TCR-peptide pair in the TAX structural group was mis-predicted as a weak binders with a binding energy deviating from the average binding energies of decoy weak binders by 0.64σ, where σ is the SD of the decoy energies. These initial results (FIGS. 4A-4B) confirm that the model is effectively able to learn the specificity rules from TCR-pMHC pairs exhibiting distinct structural representations. Moreover, RACER-m computes a continuous value capable of illustrating differences in the relative binding affinities within functional TCR-peptide clusters (FIG. 5).

While the reliable identification of strong-binding TCR-pMHC pairs is clinically useful and one important measure of model performance, simultaneous evaluation of model specificity is equally crucial for generating useful predictions on the level of a TCR repertoire. To evaluate the specificity of a global sparsity task, we next tested RACER-m's ability to discern experimentally confirmed weak-binding TCR-pMHC pairs was next tested. Peptides or TCRs were selected from the most abundant structural groups (MART-1 and TAX) in the training set to create “scrambled” TCR-pMHC pairs by cross-cluster mismatching of either TCRs or peptides. Proceeding in this manner enables a specificity test on biologically realized sequences instead of randomly generated ones. Specifically, every peptide selected from a given structural group (e.g., peptide EAAGIGILTV in the MART-1 group) was mismatched with a list of TCRs specific for peptides belonging to other groups (e.g., TAX, 1E6, and FLU) to form a set of scrambled weak binders.

Following the aforementioned testing protocols, calculated z scores for these mismatched interactions were calculated next, which were then compared to correctly matched TCR-pMHC pairs with the same peptide sequence (e.g., EAAGIGILTV). We also conducted the complementary test on TCRs using scrambled peptides. The primary advantages of this approach include (i) the ability to match amino acid empirical distributions in binding and nonbinding pairs and (ii) utilization of realized TCR sequences for specificity assessment instead of random sequences that have minimal, if any, overlap with physiological sequences.

A representative example of these tests using the MART-1 epitope and MART-1-specific TCRs is given in FIGS. 6A-6C. First, seven sets of weak binders were constructed by mismatching 36 MART-1-specific TCRs each with seven non-MART-1 peptides sampled from distinct clusters. RACER-m was applied on each weak binder to predict its binding energy and then compared this value to the distribution of decoy binding energies to obtain a binding Z score. Z scores of mismatched weak binders, together with those of correctly matched MART-1-TCR strong binders, were used to derive the receiver operating characteristic (ROC) curve (FIG. 6A). The area under the curve (AUC) was greater than or equal to 0.98 for five of the seven test sets, while the others had AUCs of 0.80 and 0.75, illustrating RACER-m's ability to successfully distinguish strong binding peptides from mismatched ones in the available MART-1-specific TCR cases.

An analogous test was performed on the five available peptide variants from the MART-1 structural group by mismatching them with 35 TCR sequences contained in the NLV, FLU, 1E6, or TAX clusters. Relative to the binding energies of correctly matched MART-1-specific TCRs, RACER-m performs well in discerning matched versus mismatched TCRs for four of the five tested MART-1 peptides (FIG. 6B), the one initial exception being peptide ELAGIGILTV. Further inspection of the TCRs in this group revealed that the TAX-specific TCR A6 (triangle sign in FIG. 6C) together with several closely associated point mutants had a z score distribution resembling that of the RD1-MART1^{High} TCR and its associated point mutants. This could be explained by the fact that the RD1-MART1^{High} TCR was engineered from the A6 TCR to achieve MART-1 specificity (31), wherein A6 was selected because of its similarity with MART-1-specific TCRs in the Va region and similar docking mode (31,32). However, the engineered (RD1-MART1^{High}) TCR is no longer specific to the TAX peptide (LLFGYPVYV, SEQ ID NO: 4), which is consistent with the z scores predicted from RACER-m. When the A6-specific TAX peptide is paired with RD1-MART1^{High} TCR, a relatively lower z score (cross sign in FIG. 6C) is predicted in comparison with the z scores from strong binders (violin shape in FIG. 6C) of the same peptide.

### Evaluation on Extended Datasets Highlights the Added Value of Structural Information

Given RACER-m's performance on the ATLAS data, we then applied the model to additional datasets to further validate its ability in the setting of global sparsity. The 10× Genomics (33) dataset details many TCR-peptide binders collected from five healthy donors. HLA-A*02:01-restricted samples in this dataset include 23 unique peptides, and the number of TCRs specific for each peptide varied from 8365 (e.g., GILGFVFTL, SEQ ID NO: 5) to 1 (e.g., ILKEPVHGV, SEQ ID NO: 6). The diversity of HLA-A*02:01 samples was substantially reduced to 1741 TCR-pMHC pairs having unique CDR3α/β and peptide sequences after removing redundancies. This large dataset was selected as a reasonable test because 89.26% of the 1741 testing pairs did not share either the same CDR3α or CDR3β sequence in common with the list of available TCR-pMHC pairs used in the training set, and 99.89% of the testing TCR-pMHC pairs did not have the same CDR3α-CDR3β combination with the training set, although 7 of the 23 peptides were shared with the training set.

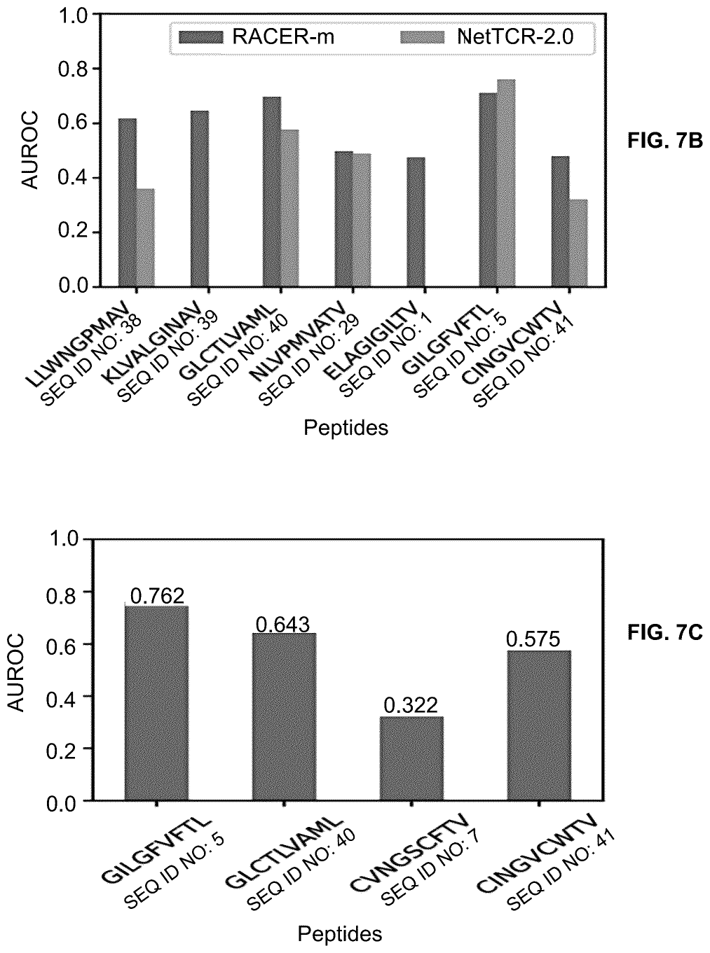

Given this relative lack of overlap with the training data, RACER-m was applied to all unique HLA-A*02:01 pairs. In a majority (88.9%) of these cases across a large immunological diversity of peptides, RACER-m successfully identifies enriched z scores in the distribution of binding TCRs (FIG. 7A). The distinction of TCRs belonging to testing versus training sets, together with the notable difference in the size of training and testing TCR-pMHC pairs, suggests that shared structural features were able to augment RACER-m's predictive power on distinct tests. Thus, the inclusion of structural information in model training enhances RACER-m's predictive ability across distinct TCR-pMHC tests.

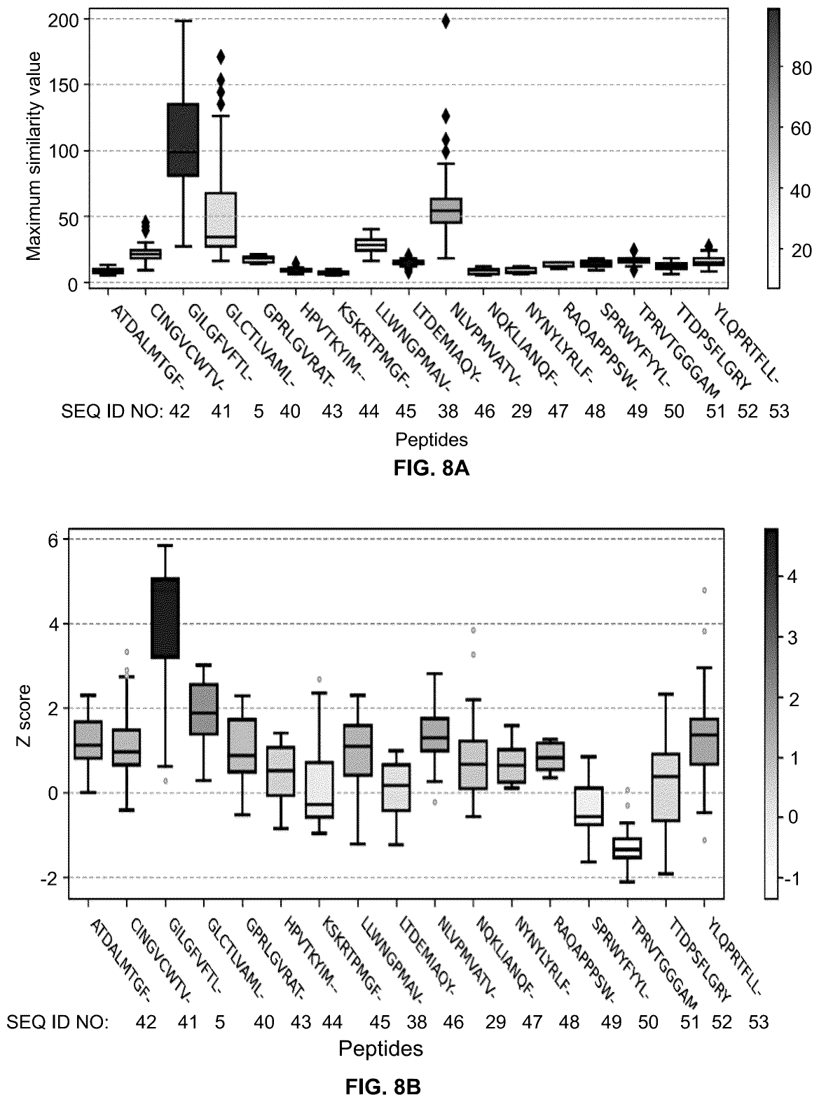

There were several cases where RACER-m's predicted distributions overlapped substantially with low z scores, indicating a failed prediction; in these cases, we investigated whether this could be explained by the lack of an appropriate structural template. A positive correlation was observed between a testing case's optimal structural template similarity and the RACER-m-predicted z scores, consistent with a decline in model applicability whenever the closest available template is inadequate for representing the TCR-pMHC pair in question. Despite this, the RACER-m approach, trained on 69 cases, was able to predict roughly 90% of strong binders contained in over 1700 distinct testing cases in the 10× Genomics dataset. A similar trend was also seen when applying RACER-m to the “global true” test set curated from the VDJdb (33) that were not included in training. RACER-m again exhibited optimal predictive performance when a reasonable structural template was available (FIGS. 8A-8B). Overall, RACER-m was able to successfully predict 56.7% of the strong binders in this set. For groups with high sequence similarities with our template structures, such as the cases of peptide GILGFVFTL (SEQ ID NO: 5) RACER-m yields a higher success rate of strong binder prediction (91.1% for cases with peptide GILGFVFTL, SEQ ID NO: 5).

RACER-m's performance was compared to NetTCR-2.0 (20), a well-established convolutional neural network model for predictions of TCR-peptide binding that is trained on over 16,000 combinations of peptide/CDR3a/p sequences. This comparison was performed on a publicly available list of TCR-pMHC repertoires curated by Zhang et al. (34), which were mutually independent of RACER-m or NetTCR-2.0 training data, wherein known strong binders and mismatched weak binders for eight unique peptides of HLA-A*02:01 were included. Because NetTCR-2.0 has a restricted length for antigen peptide (no longer than 9-mer), it cannot be applied on testing TCR-pMHC pairs with 10-mer peptides such as KLVALGINAV (SEQ ID NO: 3) and ELAGIGILTV (SEQ ID NO: 1), which are absent from the NetTCR-2.0 evaluation in FIG. 7B. The area under the ROC curve was used as a standard measure of classification success. In the majority of cases, RACER-m outperformed NetTCR-2.0 in diagnostic accuracy with higher ROC values (FIG. 7B). Last, RACER-m was further evaluated using an unrelated set of TCR-pMHC data composed of 400 samples made up of the strong binders and mismatched weak binders with four peptides and 100 TCRs (35), which also gives a good distributional performance (FIG. 7C). In one of the four peptides included in this dataset, RACER-m seems to have difficulty providing correct classification about strong and weak binders for peptide CVNGSCFTV (SEQ ID NO: 7), which could again be explained by the lack of appropriate structure templates for this pMHC and related strong binding TCRs.

### RACER-m Specificity of Point-Mutated Variants and Preservation of Local Resolvability

Encouraged by model handling of global sparsity in tests of disparate binding TCR-pMHC pairs having high sequence diversity, Next RACER-m's ability in maintaining local resolvability of point-mutated peptides with near-identical sequence similarity to a known strong binder was evaluated, which represents a distinct and usually more difficult computational problem. Understanding in detail which available point mutants enhance or break immunogenicity is directly relevant for assessing the efficacy of tumor neoantigens and T cell responses to viral evolution. In addition, the performance of structural models in accomplishing this task are a direct readout on their utility over sequence-based methods because the latter case will struggle to accurately cluster and, therefore, resolve TCR-pMHC pairs having single-amino acid differences. To evaluate RACER-m's ability to recognize point mutants, we performed an additional test on an independent comprehensive dataset of TCR 1E6 containing a point mutagenic screening of the peptide displayed on MHC. This testing set includes 20 strong binders and 73 weak binders (36), wherein strong binding to the 1E6 TCR was confirmed by tumor necrosis factor-α activity. RACER-m demonstrates enrichment of the distribution of binding energies for strong binders versus confirmed weak cases (FIG. 9A). ROC analysis (FIG. 9B) of the RACER-m's ability to resolve these groups gives an AUC of 0.78. Note that only two strong binders of this group were included in the training of RACER-m's energy model.

Inspired by these initial results on the 1E6 mutagenic screen, we extended this analysis to all point-mutated weak binding TCR-pMHC pairs in the ATLAS dataset, specifically those with K_{d} values greater than 200 μM. The results, presented template-wise for each structure in the point-mutant data, demonstrate that RACER-m improves in this recognition task when compared to NetTCR-2.0 (FIG. 9C). Last, to explicitly explore the value of structural modeling for predicting the impact of immunologically important single-amino acid differences, the predicted z scores were quantified for both strong and weak binders based on a measure of total sequence similarity. This measure was obtained by taking the maximum product of CDR3α, CDR3β, and peptide Hamming similarity between a test TCR-peptide pair and each of the training TCR-peptide pairs with an available structure. The results demonstrate that the inclusion of information from correctly identified structural templates enhances RACER-m's predictive power. Collectively, our results suggest that RACER-m offers a unique computational advantage over traditional, sequence-only methods of prediction by leveraging substantially fewer training sequences with key structural information to efficiently identify the contribution of each amino acid change.

### Discussion

Reliable and efficient estimation of TCR-pMHC interactions is of central importance in understanding and thus optimizing the adaptive immune response. The field has experienced considerable recent research activity in the development of inference-based computational methods to predict TCR-pMHC specificity (37). Decoding the predictive rules of TCR-pMHC specificity is a formidable challenge, largely owing to the extreme sparsity of available training data relative to the diversity of sequences that need to be interrogated in meaningful investigation. A majority of approaches (20,38,29) take a complementary approach to RACER-m by training on TCR and/or peptide primary sequence data alone. One recent method achieves training by relaxing a common requirement of having paired CDR3α/β sequences (38). RACER-m was developed to augment the predictive power of a relatively small number of TCR and epitope sequences by leveraging the structural information contained in solved TCR-pMHC crystal structures. The analysis focused on the most common human MHC allele variant, due to the abundance of sequence and structural data. Despite this restriction, structural heterogeneity underpinning the specificity of various TCR-pMHC pairs in distinct immunological contexts was observed. Enhancement in predictive accuracy was largely driven by the availability of a small list of structural templates, which included 66 crystal structures of TCR-pMHC complexes from the PDB.

Using the minimal list provided herein, together with mutually independent testing TCR-pMHC pairs for RACER-m and NetTCR-2.0, it was found that the model is able to outperform NetTCR-2.0 on both detection of strong binders as well as avoidance of weak binders, both representing distinct but equally important tasks. We advocate for the inclusion of such mixed performative tests for rigorous validation as a necessary and standardized component in model evaluation, in addition to model comparisons using testing data that are equally dissimilar from the training data included in competing models.

Intriguingly, incorporation of structural information into the training approach enables the development of a model that maintains predictive accuracy while dealing with both global sparsity and local resolvability, all while requiring substantially reduced training sequence data. Because of RACER-m's ability to deal with both global sparsity and local resolvability, we anticipate that this approach may be applicable to future applications that require reliable predictions on TCR responses against disparate and closely related collections of antigens. Such an approach may provide a useful theoretical tool to design, for example, tumor antigen vaccines. These results suggest that a wealth of information is contained in the structural templates pertaining to key contributors of a favorable TCR-peptide interaction, wherein conserved features across distinct TCR-pMHC pairs can be learned to mitigate global sparsity. Conversely, structural encoding of information pertinent to residues whose amino acid substitutions either preserve or break immunogenicity also assists RACER-m trained on only a small subset of all possible point mutations by identifying key contributing positions and residues, thereby preserving local resolvability.

The approach herein has been successfully applied to resolve unknown strong and weak binding TCR-pMHC pairs given those identified as such in the previously published test datasets under consideration. It is noted that perfect resolvability in the setting of repertoire-level studies that assess large numbers of randomly sampled TCR and peptide pairs would require larger z scores for distinguishing strong binders. In several test cases, our model does assign strong binders a larger score (z=4; FIG. 7A; FLU and MART-1), especially when sufficient positive training data exist. We also note that some tasks (for example, picking out single-amino acid mutants that retain strong binding) do not require competing against a large number of possible choices, and so the needed z score should be much lower.

Moreover, model accuracy correlated directly with the availability of a template having sufficient proximity to the sequences of testing TCR-pMHC pairs. As a result, we anticipate that RACER-m will improve as more structures become readily available for inclusion. Existing computational methods for identifying structural models from primary sequence data (40) may provide an efficient method of adding highly informative structures into the candidate pool for testing. This task, together with identifying the minimal sufficient number of distinct structural classes within a given MHC allele restriction, remains for subsequent investigation. The current results suggest that this is doable given the small number of structures available for explaining the diverse TCR-pMHC pairs studied herein. Notably, the inclusion of only 66 template structure augmented RACER-m's ability to accurately differentiate strong and weak binders when evaluated with hundreds and even thousands of testing TCR-pMHC pairs. This structural advantage was enhanced both by the approach of hybridizing sequence and structural information into the training and testing protocols and the availability of templates that shared sufficient sequence-based similarity to testing cases so that an adequate threading template was available.

### Example 2

### Methods for Specific Testing of the Computational Model

### Generation of Peptide:HLA-1 Monomers and Tetramers

Peptide-loaded biotinylated HLA monomers were produced via individual refolding reactions containing recombinant HLA-A*02:01, B2M, and fixed peptides (41), or recombinant HLA-A*02:01, B2M, and a UV-exchangeable peptide (42). HLA monomers were sourced from the Baylor College of Medicine Tetramer Core and Biolegend. Peptides were synthesized using standard Fmoc-chemistry and purified to >90% via LC-MS (Genscript).

Peptide:HLA-1 with the desired peptide specificities were generated via UV-mediated ligand exchange, as described elsewhere (42). Briefly, HLA monomer was diluted to 0.1 mg/ml in PBS and mixed 1:1 with 400 uM target peptide in PBS. The mixture was transferred to a polypropylene 96-well plate and exposed to 365 nm UV-C light for 30 min using a CL-3000 UV crosslinker (Analytik Jena), followed by overnight incubation at 4 ÅãC. The efficiency of UV-exchange reaction was assessed using the LEGEND MAX Flex-T Human Class I Peptide Exchange ELISA kit (Biolegend), which measures the association of biotinylated MHCI and B2M (Biolegend).

Peptide:HLA tetramers were produced by adding streptavidin-conjugates to biotinylated monomers in four serial additions, with 10-minute incubations between additions, such that the final stoichiometric ratio of streptavidin to peptide:MHCI was 1:4. Subsequently, any remaining unoccupied biotin-binding sites were quenched by the addition of 50 nM soluble D-biotin. Fluorescent tetramers were made by conjugating HLA monomers with premium-grade phycoerythrin (PE) and/or allophycocyanin (APC) streptavidin conjugates (Invitrogen). DNA-barcoded tetramers were produced by linking monomers to TotalseqGC-streptavidin conjugates (Biolegend). Conjugations were carried out on ice.

### Tetramer Staining and Flow Cytometry

TCRs were initially isolated based on their relative abundance in peripheral blood or bone marrow samples obtained from healthy donors. Cells were stained using tetramers specific for several antigens, including MART-1, FLU, and CMV-NLV, and subsequently enriched by magnetic selection to isolate tetramer-binding populations. The enriched cells then underwent approximately 10 days of in vitro expansion, using anti-CD3/CD28/CD2 stimulation supplemented with IL-2. After expansion, the cells were stained again with DNA-barcoded tetramers, each antigen specificity uniquely encoded by a distinct combination of two DNA barcodes. Tetramer-positive cells were pooled, sorted, and subsequently sequenced to identify paired TCR sequences along with their associated DNA barcodes. Cells were assigned antigen specificity based on robust binding to the correct DNA barcode combination at the single-cell level, without significant cross-reactivity. A TCR clonotype was defined as antigen-specific only if greater than 90% of cells sharing that clonotype demonstrated the same antigen specificity. These TCR sequences thus represent experimentally identified antigen-specific TCRs, rather than functionally confirmed receptors.

Cells were resuspended to 25M cells/ml in staining buffer (PBS+2% FBS+2 mM EDTA) containing 50 nM Dasatinib. Cells were stained on ice with 5 ug/ml of fluorescent and/or DNA-barcoded peptide:MHCI tetramers for 30 minutes. Cells were washed, and subsequently stained with a cocktail of antibodies including CD8a-Buv395 (RPA-T8, BD), CD3e-BV421 (OKT3, Biolegend), Dump-FITC (CD4 (RPA-T4), CD19 (HIB19), CD14 (M5E2), CD16(3G8)), anti-APC (APC003, Biolegend), anti-PE (PE001, Biolegend). In some experiments anti-streptavidin-PE (3A20.2, Biolegend) was added. For endpoint analysis cells were run on a FACS Symphony A5 (BD) and analyzed using FlowJo.

For sequencing experiments, live, tetramer-positive, dump-negative CD8 T cells were sorted. Cells were immediately run using the 10× Genomics 5′ Single Cell Immune Profiling workflow, using the manufacturer's instructions. Briefly, the sorted cells were loaded onto a lane of a Chromium Next GEM Chip G (10× Genomics) for GEM production using the Chromium controller. GEM-RT, cDNA amplification and the following library prep were using the Chromium Single Cell V(D)J Reagent Kits User Guide (v1.1 Chemistry) with Feature Barcoding technology for Cell Surface Protein (RevG) workflow (10× Genomics) as per 10× Genomics protocols.

### Illumina Sequencing

DNA libraries including Feature Barcode, TCR and GEX (gene expression) were quality controlled using TapeStation D5000 high sensitivity tape (Agilent). TapeStation bioanalyzer readout was also referenced for average DNA size calculation to determine the library loading for multiplexed sequencing. Concentration of library dsDNA was measured using a Qubit dsDNA Broad Range kit (Life Technologies, cat. Q32850). Libraries were sequenced on an Illumina NovaSeq 6000 S1 reagent kit v1.5 100 cycles (Illumina 20028319) using sequencing cycles configuration of 26+8+0+91 (Read 1+i7 Index+i5 Index+Read 2) for combined assessment of feature barcode library, TCR and GEX (RNA seq) information. Sequencing depth is 7,500 read pairs per cell for feature barcode library, 7,500 read pairs per cell for TCR library, and 20,000 read pairs for GEX library.

### Example 2

### Model Development

To predict binding affinity between TCR-peptide pairs, the previously developed RACER-m model, an energy-based biophysical framework designed to quantify TCR-pMHC interactions (12,13) was specialized. RACER-m focuses on the CDR3a and CDR3P regions as the primary mediators of context-specific TCR-peptide binding (15). The RACER-m model was retrained and optimized using 66 experimentally derived TCR-pMHC structures as the initial core structural dataset. TCR sequence used in training included 97 binder TCR-peptide pairs from the ATLAS database (23), as well as additional TCR-pMHC complexes sourced from MDACC and experimentally confirmed as binders.

Table 1 lists the total number of TCR cases (Ntotal) in each dataset and specifies their role as either training or test sets. The ATLAS dataset and MDACC_1 comprise the primary training datasets, containing known antigen specificities for MART-1, FLU, and CMV-NLV. The MDACC indices (MDACC_2 to MDACC_5) represent distinct donor and patient-derived TCR cohorts from MD Anderson Cancer Center, each sequentially evaluated as test sets.

TABLE 1
Summary of datasets

| Dataset | ηₜₒₜₐₗ | Train/Test | Source of Training Set | Antigen Specificity |
|---|---|---|---|---|
| ATLAS | 76 | Train | — | MART-1, FLU, CMV-NLV |
| MDACC_1 | 94 | Train | — | FLU, CMV-NLV |
| MDACC_2 | 118 | Test set 1 | ATLAS, MDACC_1 | All experimentally |
| MDACC_3 | 46 | Test set 2 | ATLAS, MDACC_1, 2 | confirmed (Tables 2-3) |
| MDACC_4 | 65 | Test set 3 | ATLAS, MDACC_1, 2 | |
| MDACC_5 | 295 | Test set 4 | ATLAS, DACC_1, 2, 3, 4 | |
| | | | in silico Structures | |

For each strong-binding TCR-peptide pair, an additional set of 1,000 random TCR sequences were generated to serve as non-binding examples during training. Threading these TCR-peptide pairs through candidate structural templates selected based on sequence similarity enabled the development of an optimized energy matrix for accurately predicting TCR-peptide binding affinities in this clinical context.

### Model Training and Optimization

The energy model was trained to maximize the energy gap, δE/ΔE, between binders and non-binders following a known procedure (12,13). For each binder, the average binding energy, (Ebinder), was computed, along with the mean energy of its corresponding non-binders, (Enon-binder), and the standard deviation ΔE of the non-binder energies. This approach maximizes the ratio δE/ΔE, where δE=(Enon-binder)−(Ebinder), measures the separation between binders and non-binders.

RACER-m achieves this optimization by maximizing the ratio δE/ΔE using an adapted AWSEM force field (43), a residue-level protein force field widely used for investigating protein folding and interactions (1,43). The protein-protein interaction component of the force field was utilized to compute contact energies specifically at the TCR-peptide interface. Specifically, CP atoms were employed for most residues, except for glycine, where Ca atoms were used. This calculation involves interaction weights for residue pairs and distance-based potentials to compute binding energies. The binding energy is given by:

$$E = {\sum }_{i , j} {γ}_{i , j} ( {a}_{i} , {α}_{j} ) \cdot {V}_{direct} ( {r}_{i j} ) , \qquad Eq . 6$$

where γ_{i,j}(aᵢ,aⱼ) represents interaction weights for residue pairs i and j with amino acid types aᵢ and aⱼ, respectively, and Vdirect(rij) denotes the direct interaction potential as a function of the inter-residue distance rij.

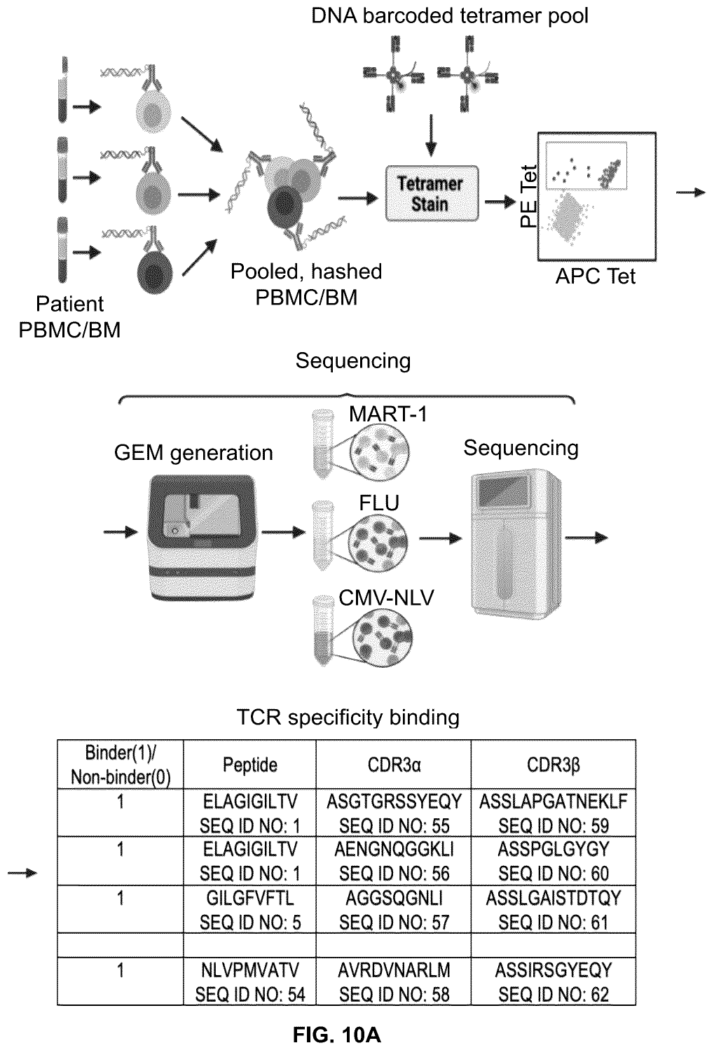

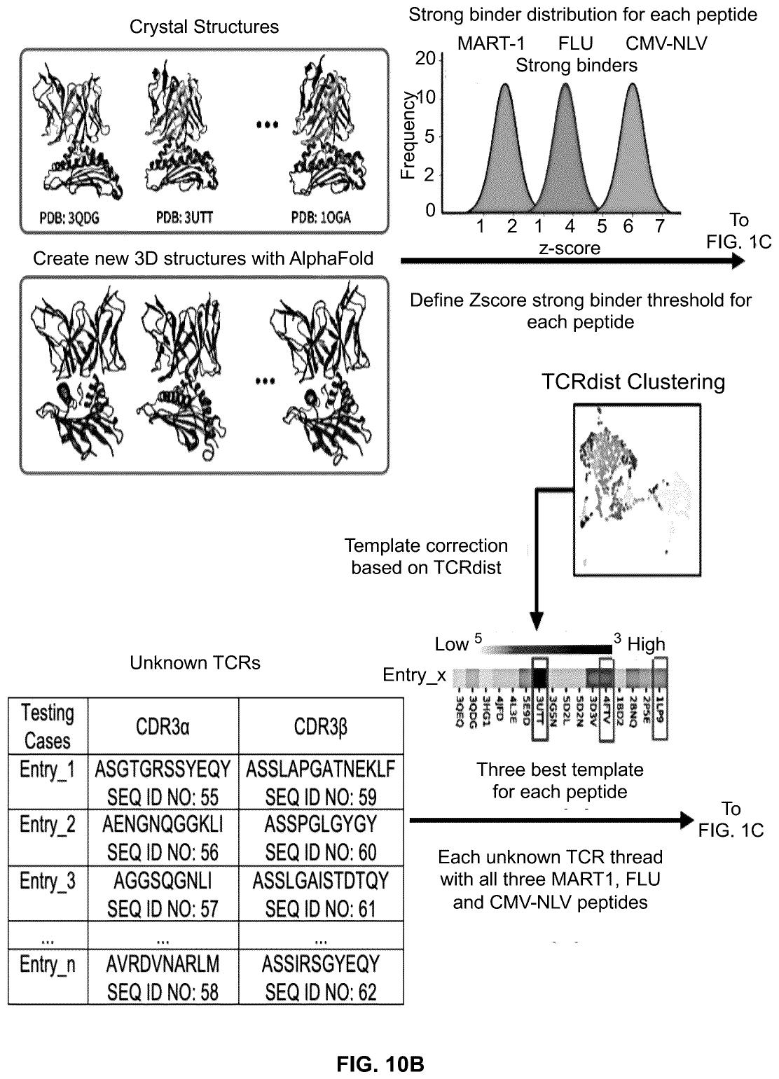

For unknown TCRs, RACER-m applies template-based modeling with MODELLER (18), matching test sequences to template structures based on composite similarity scores across CDR3α, CDR3β, and peptide regions (FIGS. 10A-10C).

### TCR-pMHC Structural Modeling

Since RACER-m evaluates binding energies based on contact interactions between peptides and TCRs, it relies on the availability of 3D structures for TCR-pMHC complexes. Obtaining this information for every test case is impractical, particularly when the relevant task involves characterizing specificity for subsets of the T cell repertoire that are distinct from training sequences. To address this limitation, RACER-m (13) utilizes MODELLER (26) to build 3D models from sequence data.

For each TCR-pMHC test pair, RACER-m computed Hamming distances separately for the peptide, CDR3α, and CDR3β sequences relative to each of the 66 template structures. Sequence similarity scores were calculated by counting the number of matching amino acids between the target and template sequences. A composite similarity score was then obtained by summing the similarity scores of the CDR3 regions and multiplying by the peptide similarity score. The template with the highest composite similarity score was selected for structure generation using MODELLER (FIG. 10B).

### Similarity-Based Template Correction

To improve the accuracy of template selection in the RACER-m model (12), a two-part correction strategy based on structural and sequence similarity measures was used. The initial RACER-m model used a training set of 66 crystal structures and organized them into clusters using the mutual Q similarity metric (19,24). Mutual Q quantifies structural similarity between 3D configurations, allowing for global comparison of two protein structures. This metric, calculated as:

$${Q}^{αβ} = \frac{2}{( N - 2 ) ( N - 3 )} {\sum }_{j > i + 2} exp [ - \frac{{( {r}_{ij}^{α} - {r}_{ij}^{β} )}^{2}}{2 {σ}_{ij}^{2}} ] \qquad Eq . 7$$

where N represents the total number of residues,

$${r}_{ij}^{α} and {r}_{ij}^{β}$$

and denote the distances between residues i and j in configurations α and ρ, respectively, and σᵢⱼ accounts for fluctuations. This method groups structures into clusters based on global structural similarities. While effective, this approach has limitations, particularly in cases where possible structural variations within diverse TCR sequences affect binding affinity.

To address these limitations, we incorporated TCRdist (44,45), a sequence-based clustering method that refines template grouping using CDR3α and CDR3β gene sequences. TCRdist computes pairwise distances between TCRs by comparing concatenated CDR loop sequences using a similarity-weighted Hamming distance. This calculation incorporates amino acid substitution penalties from the BLOSUM62 matrix (46) and applies gap penalties to account for length variation, with greater weight assigned to the CDR3 region due to its critical role in antigen specificity. This weighted metric emphasizes sequence features that are functionally important for pMHC recognition, enabling more accurate clustering of TCRs based on their positional and biochemical similarity.

### In Silico Structure Generation

In the RACER-m model, the relative scarcity of crystal structures was previously shown to limit accurate predictions on structurally disparate TCRpeptide systems (13). We will show that this limitation was especially pronounced for patients with diverse TCR clusters recognizing a specific peptide, such as MART-1, where the existing crystal structures were insufficient for accurate predictions. To address this limitation, we utilized AlphaFold3 (47) to generate additional 3D structures from a minority (˜10%) of test cases, incorporating these into the training set. Specifically, from the full set D={d1,d2, . . . , dN} of N TCRs obtained and sequenced in a given specificity experiment, we randomly select a minority fraction (N/10) for specificity determination and template construction. Following complete experimental validation of the results, this same procedure was then iterated K=6 times to establish confidence intervals for our results. This approach allowed us to assess the reliability of the generated subsets and identify the best set of structures to augment the training data, thereby improving the model's predictive performance.

In a few cases where overlap in TCR primary sequences was observed between training and test sets, new in silico derived structures were generated and evaluated for the overlapping TCRs that lacked structural information using AlphaFold3. When such sequences appeared in the randomly selected training subset, their original entries were replaced with the newly modeled structures. Predictive performance for these updated models was assessed by evaluating their ability to distinguish binding versus nonbinding cases using ROC curve analysis, repeated over multiple randomized training and test splits to ensure robustness. To comprehensively evaluate any potential effect of utilizing different methodology for in silico template creation on predictive performance, the above procedure was repeated using AlphaFold Multimer (48), along with more recent structure prediction methods, including Boltz-1 (49), which leverage large-scale transformer-based architectures and diffusion models designed for biomolecular complex prediction. These models were then evaluated as done above for AlphaFold3.

To generate new structures for the excluded cases, detailed sequence information is required, including pMHC sequences and TCR variable regions (Vα and Vφ. For the testing cases, sequence data were available only for CDR3α/β and the peptide, with the MHC being identical across all cases (HLA-A*02:01). To obtain the remaining Va/P sequences, the best template identified by RACER-m during the primary selection step was referred to. These sequences were then used to construct new 3D structures, which were incorporated into the training set to enhance model applicability. Similarly, for non-CDR regions, consensus sequences derived from curated TCR databases were selected and validated structural templates to ensure proper folding and structural stability. The complete TCR sequences, including both CDR and non-CDR regions, along with the pMHC sequences, were then used to generate new 3D structures.

### TCR-pMHC Distribution Percentile Calculation

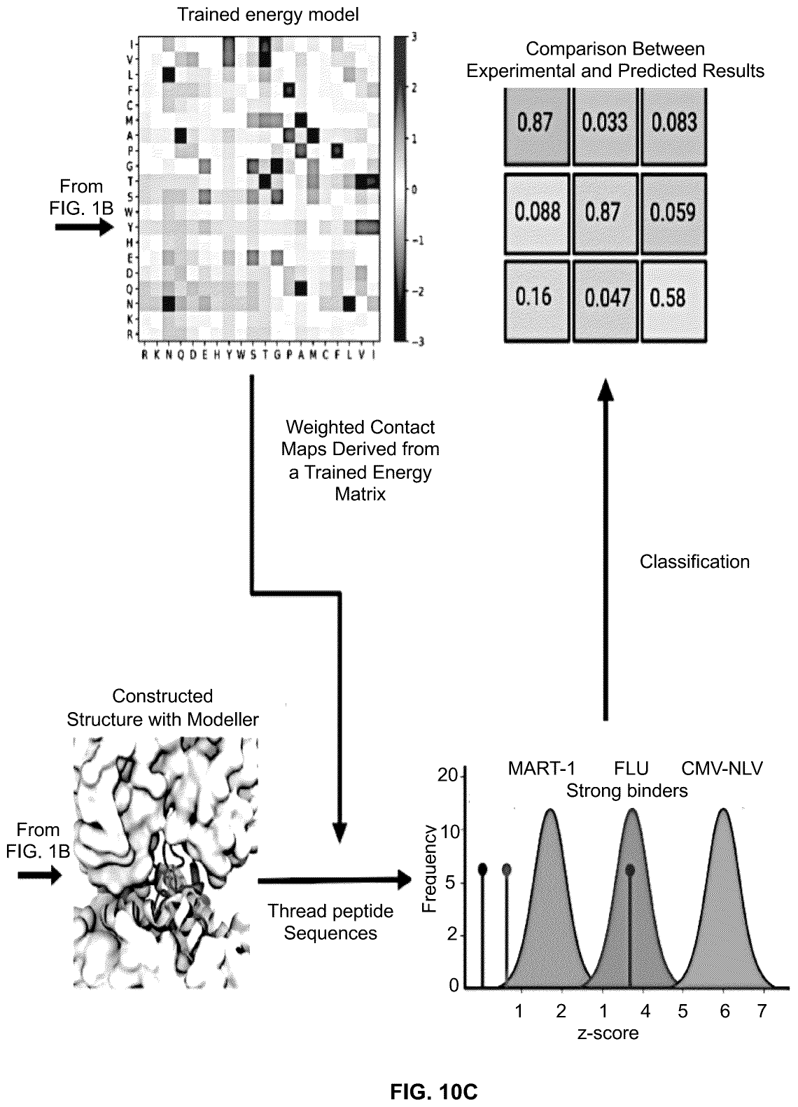

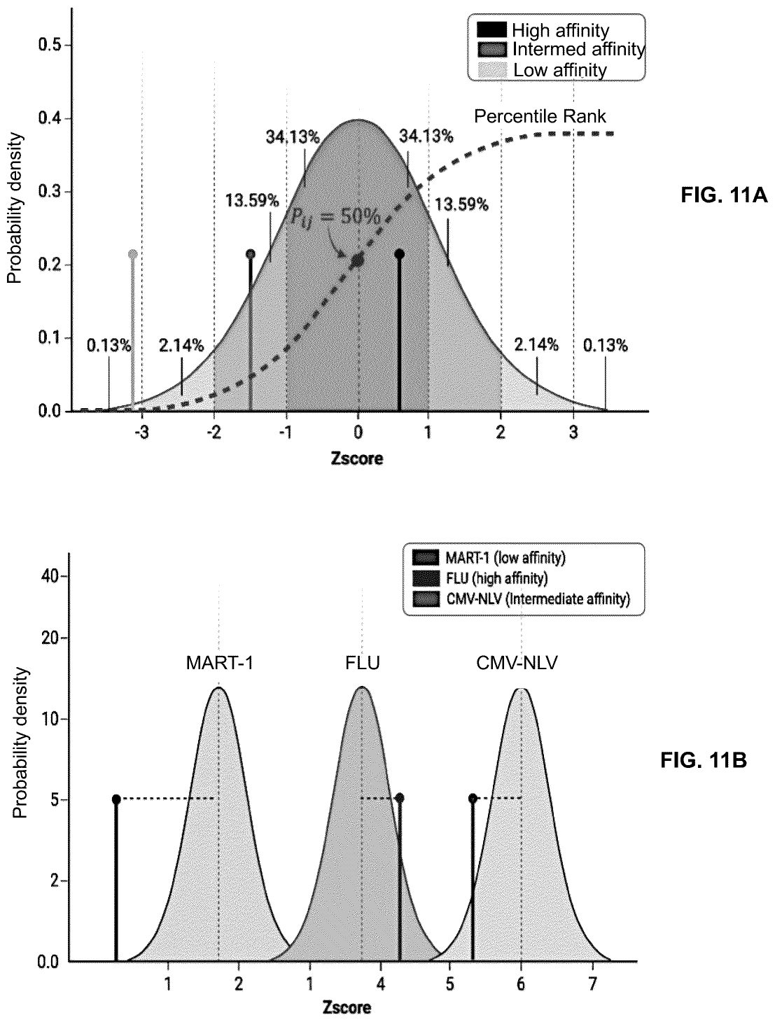

The recognition task in this study focuses on identifying T cell receptors (TCRs) as binders to a single antigen target by learning distinctive biophysical features from training distributions of binders and non-binders. A new classification method was implemented that improved upon our previous approach (RACER-m) (13) that originally classified TCR-pMHC pairs as binders based on a global Z-score cutoff. Using RACERm, Z-score distributions were generated across our training set for three antigens: MART-1, FLU, and CMV-NLV. For an unknown TCR, Z-scores were computed for each of these antigen pairs and determined the percentile ranking of each Z-score within its respective distribution. The percentile rank serves as a measure of binding potential for each peptide based on its Z-score (FIG. 11A). In this approach, the peptide having the highest percentile ranking is identified as the strongest binder. When the Z-score exceeds the mean, a higher percentile indicates a stronger binding interaction and reinforces its classification as a binder. Conversely, when the Z-score falls below the mean, larger percentile scores correspond to values closer to the mean, signifying that among the available options, this peptide exhibits the highest relative binding potential. This formulation ensures a consistent and systematic selection of the most probable binder across all scenarios.

The binding strength of each peptide is expressed as a percentile rank (Pij), which represents the percentile rank of TCR i amongst the distribution of known binder TCRs Tⱼ for peptide j out of a collection of peptides P. These distributions are characterized by their respective means (μⱼ) and standard deviations (σⱼ), derived from the training data. The percentile for each peptide is calculated using the cumulative distribution function (CDF) of the normal distribution:

$${P}_{ij} = Φ ( \frac{{x}_{i j -} {μ}_{j}}{{σ}_{j}} ) \times 100 , \qquad Eq . 8$$

where xij is the observed binding strength for TCR i in the set Tⱼ of TCRs specific for peptide j, and ϕ is the CDF of the standard normal distribution. The percentile provides an interpretable measure of the binding strength relative to the distribution, enabling meaningful comparisons among the peptides. The specificity of TCR i, Sᵢ, can be identified by maximizing over the available TCR percentiles for each peptide:

$${S}_{i} = arg {max}_{j ∈ 𝒫} {P}_{ij} . \qquad Eq . 9$$

This selection criterion associates each TCR with the peptide showing the maximal percentile binding affinity (FIG. 11B). This approach evaluates the binding strength of each TCR test case against the three peptides (MART-1, FLU, and CMV-NLV) from which sensitivity, specificity, and diagnostic accuracy are quantified. The selection criteria are designed to ensure that TCRs are classified as specific to exactly one peptide from this list.

### Example 3

### Results

### Optimized Structural Biophysical Modeling Successfully Recovers TCR Specificity.

The original RACER-m framework was specialized with the goal of generating a model capable of resolving viral (CMV-NLV, FLU) and cancer (MART-1) epitope-specific TCR sequences (13). The publicly available TCRs in the original model construction exhibited significant class imbalance, with a higher representation of MART-1-specific TCRs (n_{MART-1}=53) compared to viral (nFLu=11, n_{CMV-NLV}=12) examples. To address this imbalance and also include clinically derived cancer and viral-specific TCRs, specificity was experimentally assessed for a variety of TCRs obtained from HSCT donors and patients. In particular, the ATLAS database (23) was augmented by the addition of 20 FLU-specific and 74 CMVNLV-specific experimentally confirmed TCRs (‘MDACC_1’, Table 1).

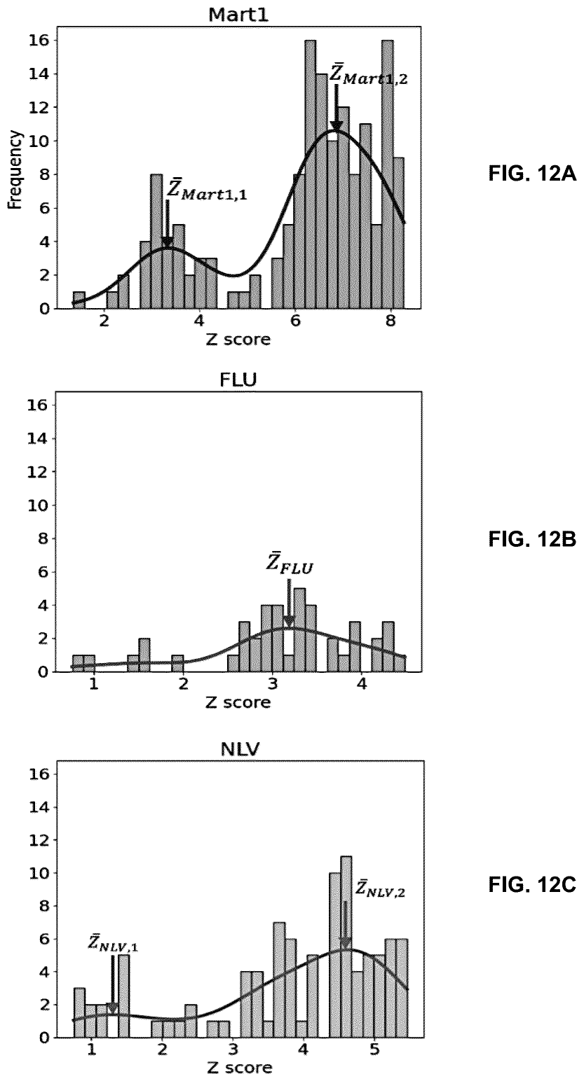

Following training and optimization, the distributions of normalized (Z-score) affinity values of TCRs specific to each of MART-1, FLU, and CMV-NLV were analyzed (FIGS. 12A-12D). To refine the selection of structural templates, the structural similarity of within-family TCRs were quantified by applying the mutual Q similarity approach (19,24). Templates that were significantly distant from others within their respective specificity groups were excluded. For example, PDB ID 5HNT, with its relatively low (3.2 Å) resolution, exhibited minimal similarity with all other MART-1-specific TCR-pMHC structures and was thus excluded. Similarly, additional templates for MART-1, FLU, and CMV-NLV were identified as outliers and removed based on mutual Q similarity as shown in Table 2. Template exclusion was performed because such outliers lead to an inaccurate estimation of percentile assignment, and their exclusion was shown to improve percentile mappings.

TABLE 2
Outlier templates removed for MART-1, FLU, and CMV-NLV

| Peptides | Removed outlier templates (PDB IDs) |
|---|---|
| MART-1 | 5NHT, 6D7G, 5E9D, 3QDJ, 6AMU |
| FLU | 5JHD, 5E6A, 5ISZ, 5EUO |
| CMV-NLV | SD2L |

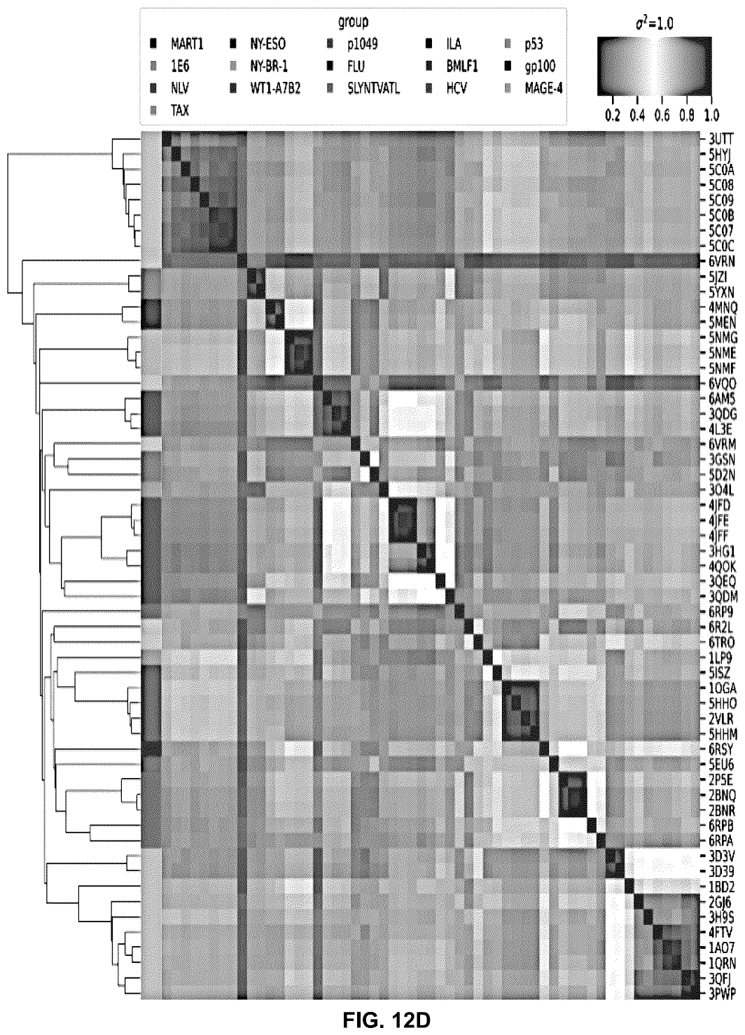

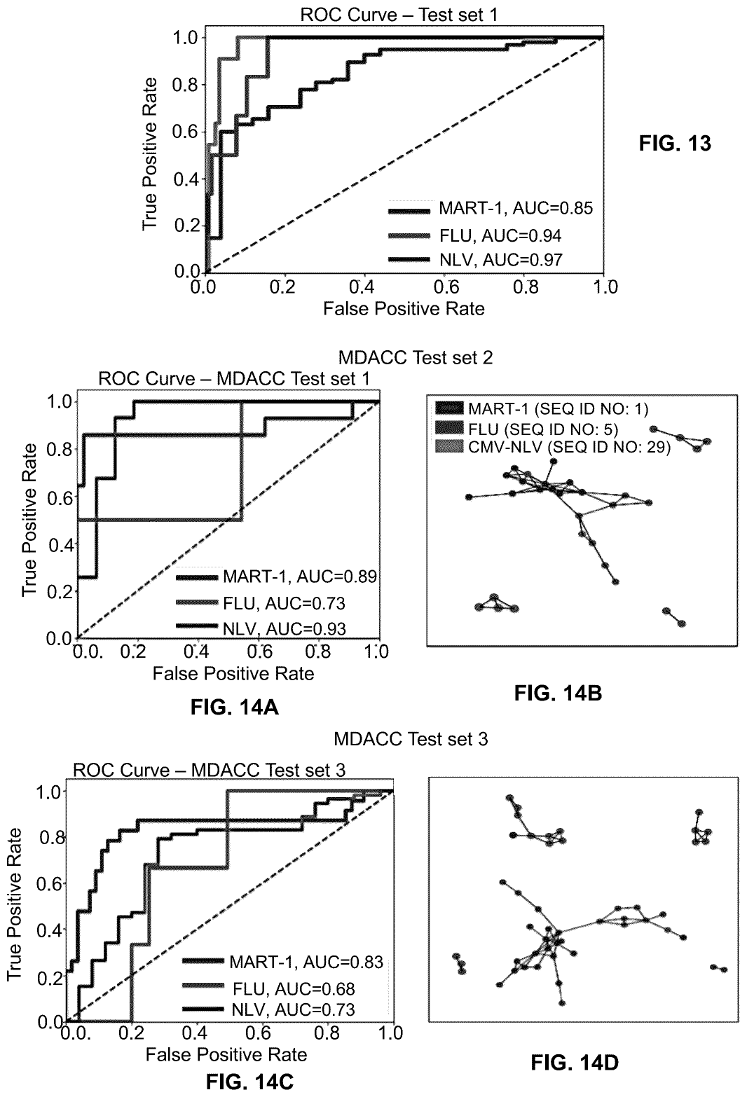

Repeating the analysis with inaccurate templates elsewhere yielded two distinct clusters for MART-1, one cluster for FLU, and two clusters for CMV-NLV, as visualized by hierarchical clustering (FIG. 12D). Consequently, distinct statistical tests corresponding to each structural cluster based on the identified Z-score distributions were established. MART-1 cases exhibited two empirical distributions with means of μ_{MART-1,1}=3.2 and μ_{MART-1,2}=6.8. CMV-NLV cases also displayed two distinct distributions with means of μ_{CMV-NLV,1}=1.3 and μ_{CMV-NLV,2}=4.6. In contrast, FLU cases were characterized by a single distribution with a mean of μ_{FLU}=3.2. All distributions were normalized to a standard deviation of σ=1 (FIGS. 13A-13D). These distributions were integrated into the model by assigning each test case to the appropriate structural cluster using sequence-based similarity to the closest template. Because each structural cluster corresponded uniquely to one statistical distribution, the respective statistical test was then performed by calculating the within-cluster percentile rank. The resulting percentile rank thus quantified the relative binding strength of the TCR to each pMHC complex for the three target peptides analyzed.

To evaluate optimized model performance, the specificity of 118 HLA-A*02-restricted TCRs was predicted from an independent PBMC and BM-derived TCR cohort (MDACC_2, Table 1). The ROC curves for MDACC_2 are summarized in FIG. 13, while Table 3 reports the sensitivity, specificity, and diagnostic accuracy. The model demonstrated strong predictive performance across all three peptides, with the highest accuracy for CMV-NLV (0.91), followed by FLU (0.90) and MART-1 (0.85). Moreover, this refined template selection using mutual Q similarity contributed to improving classification performance, particularly by ensuring that the structural representations used for MDACC_2 cases were well aligned with the training distributions (FIG. 12D).

TABLE 3
Specificity, Sensitivity and Accuracy for
MART-1, FLU and CMV-NLV using MDACC_2

| Peptide | Specificity | Sensitivity | Accuracy |
|---|---|---|---|
| MART-1 (n = 97) | 0.92 | 0.85 | 0.85 |
| FLU (n = 9) | 0.95 | 0.88 | 0.9 |
| CMV-NLV (n = 12) | 0.96 | 0.89 | 0.91 |

### Sequence Similarity-Based Clustering Enables Appropriate Template Selection in Testing

Given the model's initial predictive performance, an updated policy was developed whereby the specificities of previously obtained donor-derived TCR sequences, once experimentally confirmed, can be incorporated into model training. A series of repeated tests of the modeling framework was performed on additional independent datasets. The prior training and experimentally confirmed test data were incorporated into an updated training dataset (comprised of ATLAS, MDACC_1, and MDACC_2 sequences). Two additional independent test datasets (MDACC_3 and MDACC 4) from PBMC and BM-derived TCR cohorts were then used to assess the model's ability to generalize across diverse TCR repertoires. In all subsequent test cases, any duplicate TCR sequences were removed from predictions.

In the initial test (MDACC 2), a structure-based template selection approach effectively mapped test sequences with an appropriate structure. However, when this same method was applied to additional unseen TCR cohorts (MDACC_3 and MDACC_4), the initial approach failed to maintain predictive accuracy. A careful examination of the inverse mapping (f⁻¹:Y→X) from the test TCR sequences Y to the set of structural templates available in training X revealed sensitivity issues, wherein test sequences mapped to multiple templates in some cases, while in others, similar test sequences were mapped to disparate structures for threading. As a result, TCRdist (44,45), a previously established sequence-based clustering method that leverages detailed sequence similarity metrics to partition TCR sequences into distinct clusters was utilized. Specifically, an optimized clustering threshold was defined by selecting the largest TCRdist radius around an antigen-associated (centroid) TCR for which fewer than one in one million (10⁻⁶) background TCRs are expected to fall. This threshold was determined by constructing empirical cumulative distribution functions for each candidate centroid TCR using both antigen-associated sequences and a specifically constructed background repertoire comprising V-J genematched synthetic sequences and cord blood TCRs. By selecting this threshold, the capture of closely related antigen-associated TCRs was maximized while minimizing the inclusion of nonspecific, background TCR sequences. Although this approach inevitably groups together sequences with subtle structural variations, it strikes a balance between sensitivity, capturing relevant TCR sequences, and specificity excluding unrelated background sequences.

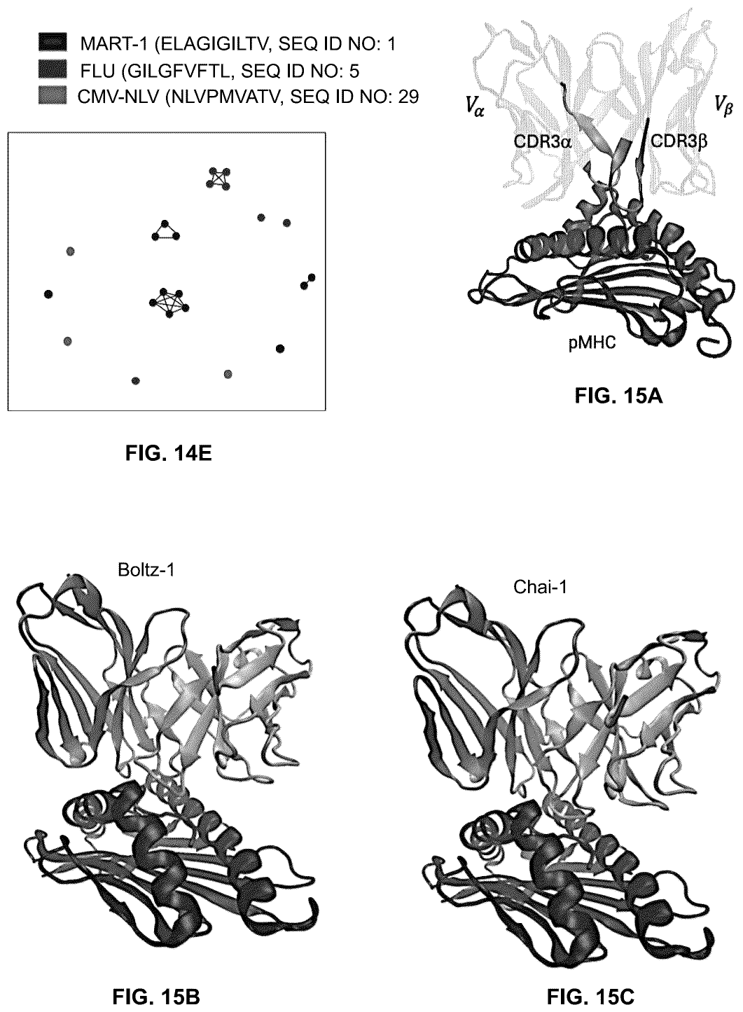

TCRdist clustering was performed on the updated training set (ATLAS, MDACC_1, and MDACC 2) using CDR3α/β sequences and genetic features (J-gene and V-gene), available from the Immune Epitope Database (IEDB) (50) and germline sequenced MDACC datasets (excluding ATLAS sequences lacking J-gene and V-gene information). This clustering was then used to restrict candidate structural templates to those representing multiple closely related TCR sequences. Applying TCRdist to MDACC_3 and MDACC_4 refined template selection by filtering out singleton clusters. This method improved template selection for MART-1, FLU, and CMV-NLV, ensuring a prioritized list of structural templates accurately reflected the diversity of TCR sequences (FIG. 14E).

Using the updated TCRdist criteria for template matching, the model was retrained and tested using the new test datasets. These predictions were then compared against experimentally determined ground-truth specificity for each TCR, which was also used to assess clustering accuracy within the TCRdist framework. The ROC analysis demonstrated that the model effectively distinguished binder TCRs across different epitope groups. The AUC values for MART-1 were 0.89 and 0.83 for MDACC_3 and MDACC_4, respectively. For FLU, the corresponding values were 0.73 and 0.68, respectively, while for CMV-NLV, the model achieved AUC scores of 0.93 and 0.73, respectively. Notably, the limited number of FLU cases in MDACC_3 and MDACC_4 (only two cases each) may have influenced the lower reliability of the FLU predictions in these datasets. FIGS. 14A and 14C present the ROC curves for MDACC_3 and MDACC_4, while FIGS. 14B and 14D illustrate the corresponding TCRdist-based clustering results, with specificities indicated following experimental confirmation. In these clustering representations, clusters containing singleton TCRs were filtered for visualization purposes but remained included in the overall analysis. Moreover, these clustering results confirm that the structural families present in the training set (FIG. 15E) adequately cover the TCR diversity observed in MDACC_3 and MDACC_4.

These results were compared with an alternative classification strategy that focused solely on utilizing sequence based classification of test TCRs based on TCRdist alone, thereby foregoing any structural modeling. While TCRdist provides a powerful clustering method based on sequence similarity, it alone is insufficient for accurate classification, there are numerous misclassifications among MART-1, FLU, and CMV-NLV peptides when relying solely on TCRdist clustering (FIGS. 14B, 14D). Precision (TP/(TP+FP)), Recall (TP/(TP+FN)), and Accuracy ((TP+TN)/(TP+TN+FP+FN)) were calculated for both methods and summarized in Table 3. The results demonstrate that structural modeling combined with TCRDist-based template selection consistently achieves higher classification performance.

TABLE 4
Precision, recall and accuracy for MART-1, FLU and CMV-NLV using
TCRdist and RACER-m methods on Test Set 2 and Test Set 3

| | | Precision | | Recall | | Accuracy | |
|---|---|---|---|---|---|---|---|
| | | | RACER + | | RACER + | | RACER + |
| Test Sets | Peptides | TCRdist | TCRdist | TCRdist | TCRdist | TCRdist | TCRdist |
| Test Set 2 | MART-1, | 0.55 | 1 | 0.84 | 0.76 | 0.71 | 0.93 |
| | n = 13 | | | | | | |
| | FLU, | 0. | 0.4 | 0. | 1 | 0.56 | 0.89 |
| | n = 2 | | | | | | |
| | CMV = NLV, | 0.62 | 0.93 | 0.7 | 0.87 | 0.52 | 0.84 |
| | n = 31 | | | | | | |
| Test Set 3 | MART-1, | 0.44 | 0.71 | 0.84 | 0.75 | 0.63 | 0.72 |
| | n = 13 | | | | | | |
| | FLU, | 0. | 0 | 0.5 | 0 | 0.98 | 0.88 |
| | n = 2 | | | | | | |
| | CMV = NLV, | 0.62 | 0.78 | 0.74 | 0.7 | 0.78 | 0.72 |
| | n = 31 | | | | | | |

### Incorporation of in Silico-Derived TCR-pMHC Structures Generalizes Model Predictions to Diverse and Unseen TCR Test Sequences

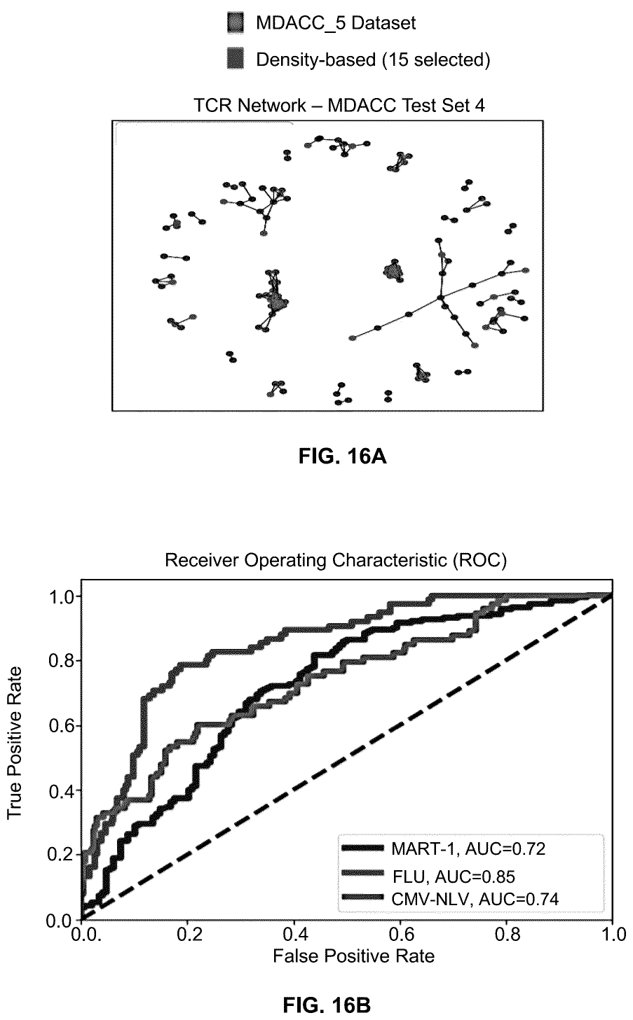

A final test dataset was used for further evaluation comprising more patient derived PBMC and BM-derived TCRs than all previous test sets combined (‘MDACC_5’, Table 1). TCR clustering revealed that this test dataset was substantially more diverse and comprised of disparate TCR sequences relative to the previous cases (FIG. 16A). The hypothesis is that this diversity would limit predictive efficacy in the existing framework, and that the inclusion of an informed choice of additional templates would lead to improved model predictions.

To assess this possibility, a small (n=15; ˜5%) subset of maximally informative TCRdist-clustered test TCR sequences were identified for the creation of new structural templates. Specifically, TCRs from clusters containing four or more members were selected and deliberately excluded those from highly dense ones (particularly the two central clusters in FIG. 16A). The rationale for this exclusion was based on the observation that densely populated clusters typically reflect sequences with limited variability and conserved, canonical motifs that are shared across individuals. These high similar TCR sequences tend to have conserved structural conformations, and as such, their structural space is likely well represented by existing crystal structures. By excluding these clusters from the selection, put focus on less populated, more heterogeneous cases that may harbor underexplored structural configurations (10,44).

Once identified, each case was a priori experimentally tested to confirm peptide specificity and subsequently excluded from predictive assessment. These confirmed TCR-pMHC systems were used to create additional in silico derived structures using AlphaFold3 (47). In each instance, sequence data were available only for CDR3α/β and the corresponding peptide; accordingly, the remaining Vα/β sequences from the closest available RACERm-identified crystal structure were incorporated along with HLA-A*02:01 to generate synthetic TCR-pMHC structures for these selected cases (FIGS. 15A-15C). FIG. 16A illustrates the selection process, where gray nodes represent MDACC_5 test TCRs, and pink nodes indicate the chosen cases for in silico structure generation and subsequent inclusion in the training set.

The ROC curve in FIG. 16B illustrates the performance of this selection procedure, yielding ROC-AUC values of 0.72, 0.85, and 0.74 for MART-1, FLU, and CMV-NLV, respectively. A repeat attempt to create these predictions in the absence of new structures resulted in significant reductions in overall predictive performance, thereby quantifying the added benefit of utilizing an update strategy requiring minimal additional TCR information.

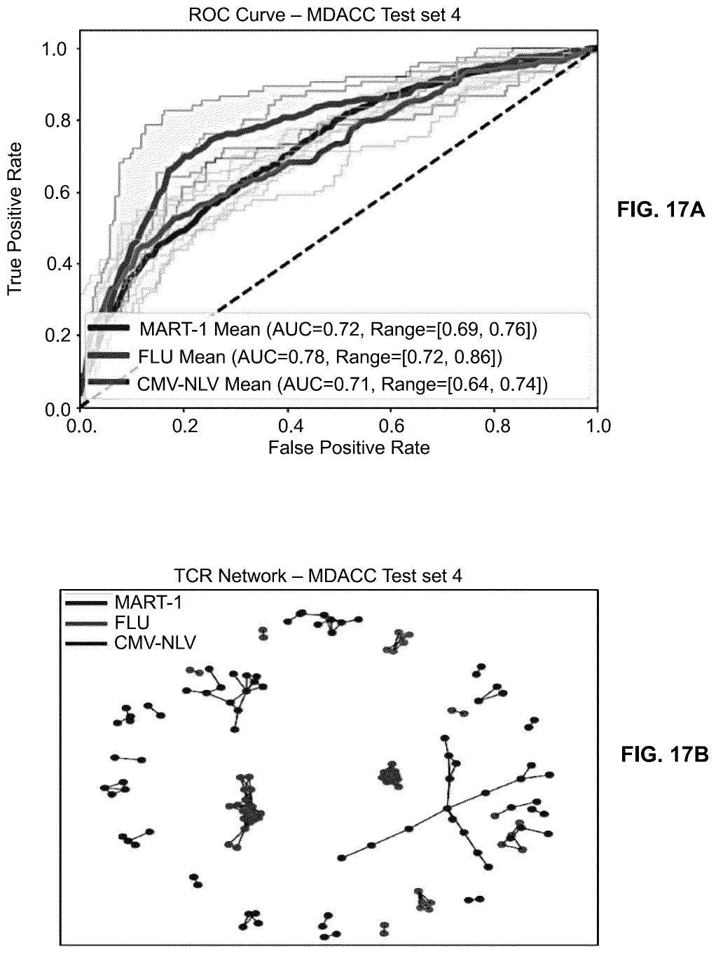

The above results were also compared with two alternative approaches. In the first alternative approach, a randomized procedure was utilized in place of the original densitybased method to identify a subset (10%) of TCRs from the MDACC_5 test set for structure generation. This was followed by predictive assessment on the 90% hold-out cases as a consistency check. The RACER-m model was retained using the same training procedure as before while incorporating the new structural templates. The optimized model was subsequently evaluated on each of the test datasets. This strategy effectively resolved duplication between training and testing sets while enriching the training data with structurally informative cases. This random selection was repeated over six iterations to establish confidence intervals on the ROC curves derived from randomized template selection (FIG. 17A). The repeat analysis following the generation of new structures for overlapping TCR sequences between training and test sets resulted in notable improvements in predictive performance. The ROC values across the different random subsets ranged from 0.69 to 0.76 for MART-1, 0.72 to 0.86 for FLU, and 0.64 to 0.74 for CMV-NLV, all of which were comparable to the performance of the original density-dependent selection criterion.

To further assess the influence of these generated structures, mutual Q heat maps were compared for the highest- and lowest-performing selections. In the best-performing heat map, for MART-1 structures are learned that the experimental crystal structures form a distinct family, while the in silico structures form a separate, non-overlapping group. In contrast, the poorest-performing heat map shows that, within the same family, the in silico and experimental structures share significant structural similarity, indicating that the additional in silico structures do not effectively expand the model's diversity of structural families available for predictions. The experimentally confirmed TCRdist-clustered specificities (FIG. 17B) illustrate the relative sequence diversity in MDACC_5. In particular, MART-1 exhibits highly diverse TCR clusters, whereas FLU andCMV-NLV display patterns of limited and dense clusters. This result further reinforces the importance of including meaningful structures into the training and optimization steps to appropriately generalize the modeling frameworks to new test data.

Both of the above approaches did not explicitly take into account sequence similarity of test cases relative to existing crystal structures. To evaluate the importance of incorporating new templates that are distinct from existing ones on prediction accuracy, a third template selection approach was implemented. First identified were clusters that already contained experimental crystal structures (red nodes) and then prioritized TCRs (light blue nodes) from clusters lacking existing structures for structure generation. The same density-dependent selection of (n=15) ˜5% of the MDACC_5 TCRs were repeated to a priori confirm their specificity. The model was then retrained and tested on the remaining data. This approach led to a slight improvement of ROC-AUC for CMV-NLV, as the relative dearth of available experimental structures for this case provided an opportunity for in silico-derived structures to enhance model ability. In contrast, for MART-1 and FLU, the refined selection yielded results similar to those obtained with the (n=30) random selection strategy and the (n=15) representative case selection informed by cluster density and size (FIGS. 16A-16B, FIGS. 17A-17B), indicating that the structural selections were largely consistent across all strategies.

Lastly, to evaluate the robustness of the approach to any particular choice of in silico structure creation method, the analysis was repeated again, this time utilizing several alternative approaches, including AlphaFold3 (14), AlphaFold Multimer (30), and Boltz-1 (49). Repeat predictive assessments demonstrated that overall model performances across these methods were comparable, achieving average ROC-AUC scores for the MDACC_5 test set of 0.72, 0.78, and 0.71 (AlphaFold3), 0.74, 0.76, and 0.73 (AlphaFold Multimer), and 0.70, 0.76, and 0.72 (Boltz-1) for the Mart1, Flu, and NLV peptides, respectively. These results highlight that the computational approach is robust to any particular method of structure generation, provided that sufficient structural diversity and quality are maintained. Collectively, these results demonstrate the effectiveness of utilizing a small number of structurally informed templates to enhance model generalization to previously unseen TCRs. Additionally, iterative updating with the inclusion of relevant in silico-derived structural data can effectively augment the training of the RACERm model, particularly in scenarios where experimental structural data are limited.

### DISCUSSION

TCR-pMHC prediction is a formidable task, owing both to the diversity of sequence space, together with a sparse sampling of that space in any feasible experimental approach. The aim was to reliably discern TCR specificity against tumor and viral epitopes in the setting of donor-derived hematopoietic stem cell transplant. Model applications are wide ranging and include, in the context of transplant, identifying tumor antigen-specific TCRs from those that expand during viral reactivation. Thus a biophysical modeling framework was developed to appropriately cluster and assign TCR specificity, in addition to an update policy for improving the model as continuous additional patient TCR sequences become available and ultimately validated.

One primary advantage of proceeding with a structure-based approach is the extent to which biophysical interactions can be reliably learned at the level of pairwise TCR-peptide amino acid interactions, which then enable training on a relatively small set of sequences and structures. In this context, patient-derived TCR sequence provide an additional opportunity to refine this approach. The initial training and predictions benefited from structural clustering to identify antigen-specific TCR clusters corresponding to unique peaks in the distributions of binding interactions, indicating that the RACER-m model performs well in datasets with less diverse TCR repertoires when appropriate structural templates are available. However, test datasets with significant TCR diversity required 1) additional sequence-based clustering and 2) the inclusion of further in silico-derived structures for optimal predictions. The first need arose either from an ill-defined mapping of test sequences to multiple template structures or from poor assignment of closely related test sequences to disparate structural templates.

To address these issues, additional structural templates generated by AlphaFold3 were incorporated, which expanded the structural diversity within the training set and ensured better coverage of TCR clusters observed in diverse patient datasets. The TCRdist approach effectively captures sequence variations not apparent from structural similarities alone, particularly when templates are limited or TCR diversity is high. Consequently, by integrating these new templates, model predictions became more robust. It is noted that the combination of TCRdist assignment and structural model prediction is required for maximal accuracy, where the use of either method to independently assign TCR specificity results in reduced predictive accuracies. These improvements are further supported by the performance of randomly selected training subsets, with the best selection yielding a significantly improved ROC curve due to broader coverage of TCR clusters. Notably, the results suggest that the RACERm framework maintains predictive utility over a range of structure generation methods, which is reassuring from the standpoint of consistency across structure type.

In the analysis of datasets MDACC_2, MDACC_3, and MDACC_4, a strategy of identifying a minimal yet representative subset of structural templates capable of capturing TCR specificity was pursued initially. In contrast, the local diversity of MDACC_5 limited the ability to reliably predict specificity using a fixed set of templates. This finding motivated the inclusion of broader structural coverage to improve model generalizability. While these two strategies contribute opposing effects on the number of available templates, they complement one another meeting the shared objective of selecting structural templates that best represent the distribution of TCRs within each dataset. Despite the above strategy, not all included structures contributed positively to predictive accuracy. Templates exhibiting suboptimal quality or limited representativeness of TCR clusters correlated with poorer performance outcomes. Specifically, an association between poorly performing templates and unfavorable Z-score distributions was observed, suggesting that structural inadequacy, such as inaccurate modeling or poor alignment with real TCR-peptide binding conformations, can distort energetic predictions. These findings emphasize the importance of carefully selecting structural templates to balance coverage and quality, and highlight that both template diversity and accuracy are critical for improving model generalizability across patient cohorts.

The analysis identified several variations in update procedures whereby new in silico-derived structures can be incorporated for more accurate future predictions, which were primarily partitioned based on randomized vs. directed choice of new structural templates selected from diverse sequence clusters. While the strategies explored here offered comparable predictive performance, each case possesses different implications on updating with respect to new information. For example, randomization, while requiring more upfront specificity data, can be used as an unbiased method for incorporating new structures in sequence. In contrast, the selection-based approach, while more efficient in requiring minimal TCR specificity, may result in suboptimal template selection relative to future TCR datasets, particularly if the distribution of sequence diversity becomes skewed in favor of a cluster for future test TCRs. In reality, the best choice is likely context-specific and requires further experimental validation supporting the initial findings herein. This approach, while achieving high predictive accuracy, still required experimental confirmation of a small sub-sampling of test cases. The principal advantage in applying our structural model is that a few carefully selected templates with sufficient diversity can recover accurate predictions on a majority of unseen test cases.

The improved predictive accuracy of the RACER-m model has important clinical implications for allo-HSCT. By reliably identifying GVL-specific TCRs, clinicians can better select donor-derived TCR repertoires that maximize anti-leukemia effects while minimizing off-target expansions that may lead to graft-versus-host disease (GVHD) or responses to viral antigens. This capability may help refine donor selection and guide therapeutic interventions to improve patient outcomes in allo-HSCT. It is contemplated that future studies could explore expanding the RACER-m framework by incorporating a broader range of peptide targets beyond those studied here. This augmented set could include a larger collection of putative tumor-associated antigens. Additionally, the inclusion of minor-histocompatibility differences (SNP polymorphisms) resulting in differences in self and donor-derived antigens could in the future enable clinicians to add the additional criterion of minimization of GVHD in the optimal selection of a donor TCR repertoire.

### REFERENCES

1. Zhang et al. Science advances, 7(20):eabf5835, 2021.
2. Grant et al. Journal of Biological Chemistry, 291(47):24335-24351, 2016.
3. George, et al. Proceedings of the National Academy of Sciences, 114(38):E7875-E7881, 2017.
4. Chau, et al., Physical Review E, 106(1):014406, 2022.
5. Meynard-Piganeau, et al., Proceedings of the National Academy of Sciences, 121(24):e2316401121, 2024.
6. Kwee, et al., bioRxiv, pages 2023-04, 2023.
7. Ghoreyshi, et al. Biophysical Journal, 2024.
8. Teimouri, et al., Frontiers in Immunology, 15:1510435.
9. Montemurro, et al., Communications biology, 4(1):1060, 2021.
10. Glanville, et al., Nature, 547(7661):94-98, 2017.
11. Zahra S Ghoreyshi and Jason T George. Frontiers in Immunology, 14:1228873, 2023.
12. Lin, et al., Nature Computational Science, 1(5):362-373, 2021.
13. Wang, et al., Science Advances, 10(20):eadl0161, 2024.
14. Lin et al. Nat. Comput. Sci. 1:362-373 (2021).
15. La Gruta et al. Nat. Rev. Immunol. 18, 467-478 (2018).
16. Davtyan et al. J. Phys. Chem. B 116, 8494-8503 (2012).
17. Zheng et al. Proc. Nat/. Acad. Sci. U.S.A. 109, 19244-19249 (2012).
18. B. Webb and A. Sali, Curr. Protoc. Bioinformatics 54, 5-6 (2016).
19. Chen et al. J. Phys. Chem. B 121:3473-3482 (2017).
20. Montemurro et al. Commun. Biol. 4:1060 (2021).
21. R. Gowthaman and B. G. Pierce, Bioinformatics 35, 5323-5325 (2019).
22. Vita et al. Nucleic Acids Res. 47, D339-D343 (2018).
23. Borrman et al. Proteins 85, 908-916 (2017).
24. Cho et al. Proc. Nat/. Acad. Sci. U.S.A. 103:586-591 (2006).
25. H. M. Berman, Nucleic Acids Res. 28, 235-242 (2000).
26. George et al. Proc. Natl. Acad. Sci. U.S.A. 114:E7875-E7881 (2017).
27. Chau et al. Phys. Rev. E 106:014406 (2022).
28. R. Gowthaman and B. G. Pierce, Methods Mol. Biol. 2120:197-212 (2020).
29. Rudolph et al. Annu. Rev. Immunol. 24:419-466 (2006).
30. B. G. Pierce and Z. Weng. Protein Sci. 22:35-46 (2013).
31. Smith et al. Nat. Commun. 5:5223 (2014).
32. Pierce et al. PLOS Comput. Biol. 10:el003478 (2014).
33. 10× Genomics, Tech. Rep., 10× Genomics, 2019.
34. Zhang et al. Sci. Adv. 7:eabf5835 (2021).
35. Grant et al. J. Biol. Chem. 291:24335-24351 (2016).
36. Bulek et al. Nat. Immunol. 13:283-289 (2012).
37. Z. S. Ghoreyshi and J. T. George. Front. Immunol. 14, (2023).
38. Meynard-Piganeau et al. bioRxiv 549669 [Preprint], 2023.
39. Kwee et al. bioRxiv 538237 [Preprint], 2023.
40. P. Bradley, eLife 12:e82813 (2023).
41. Garboczi, et al., Proceedings of the National Academy of Sciences, 89(8):3429-3433, 1992.
42. Rodenko, et al. Nature protocols, 1(3):1120-1132, 2006.
43. Davtyan, et al. The Journal of Physical Chemistry B, 116(29):8494-8503, 2012.
44. Dash, et al., Nature, 547(7661):89-93, 2017.
45. Mayer-Blackwell, et al. Elife, 10:e68605, 2021.
46. Steven Henikoff and Jorja G Henikoff. Proceedings of the National Academy of Sciences, 89(22):10915-10919, 1992.
47. Abramson, et al., Nature, pages 1-3, 2024.
48. Evans, et al. biorxiv, pages 2021-10, 2021.
49. Wohlwend, et al. bioRxiv, pages 2024-11, 2024.
50. IEDB. Immune epitope database and analysis resource, 2025. Accessed: 2025-01-19.
51. Zhang, et al. Computational and Structural Biotechnology Journal, 23:165-173, 2024.
52. Moris, et al. Briefings in bioinformatics, 22(4):bbaa318, 2021.
53. Weber, et al. Bioinformatics, 37(Supplement_1):i237-i244, 2021.

What is claimed is:

1. A computer-implemented method for training a computational model to predict T cell-antigen specificity, comprising:
a) inputting into the computational model a data training set of known T cell-antigen interactions;
b) generating an optimized description of amino acid interactions from the data set; and
c) testing the predictive ability of the computational model and the optimized description against known T cell-antigen pairs.

2. The computer-implemented method of claim 1, further comprising updating the computational model via the steps of:
d) inputting an updated data training set into the computational model; and
e) generating an updated description of the optimized description of amino acid interactions from the updated data training set; and
f) testing the predictive ability of the computational model and the updated optimized description against known T cell-antigen pairs.

3. The computer-implemented method of claim 2, further comprising repeating at least once steps d), e) and f).

4. The computer-implemented method of claim 1, wherein the data training set comprises a sparse sampling of the known T cell-antigen interactions.

5. The computer-implemented method of claim 4, wherein the sparse sampling of the known T cell-antigen interactions comprises a plurality of human MHC-I allele variant peptide sequences and protein crystal structures thereof.

6. The computer-implemented method of claim 5, wherein the human MHC-I allele variant is HLA-A*02:01.

7. The computer-implemented method of claim 1, wherein the optimized description of amino acid interactions is a combined sequence-structural model of T cell receptor-pMHC specificity that comprises biophysical information.

8. A computer implemented method for predicting T cell specificity against antigens, comprising:
a) training a computational model for T cell-antigen specificity prediction using a data set of known T cell-antigen interactions to generate an optimized energy model of amino acid interactions that determine T cell specificity against antigens; and
b) using the trained computational model and the optimized energy model to predict which T cells recognize an unknown antigen or which unknown T cells recognize a given antigen.

9. The computer implemented method of claim 8, further comprising refining the computational model via the steps of:
c) incorporating a new data set of known T cell-antigen interactions to train an updated computational model to generate an updated optimized energy model of amino acid interactions that determine additional T cell specificity against additional antigens; and
d) using the updated trained computational model and the updated optimized energy model to predict which of the additional T cells recognize an unknown antigen or which unknown T cells recognize a given additional antigen.

10. The computer-implemented method of claim 9, further comprising repeating at least once steps c) and d).

11. The computer-implemented method of claim 8, wherein the data training set is a sparse data set of the known T cell-antigen interactions.

12. The computer-implemented method of claim 11, wherein the known T cell-antigen interactions comprise a plurality of human MHC-I allele variant peptide sequences and protein crystal structures thereof.

13. The computer-implemented method of claim 12, wherein the human MHC-I allele variant is HLA-A*02:01.

14. The computer-implemented method of claim 8, wherein the trained computational model and the optimized energy model resolve the T cell-antigen specificity of the unknown T cell receptors against tumor antigens and viral antigens.

15. The computer-implemented method of claim 8, wherein the optimized energy model is utilized for predicting binding specificity of an antigenic peptide toward a specific T cell receptor based on the respective peptide sequences thereof.

16. A system to predict T cell-antigen specificity comprising a computational model tangibly stored on an electronic device having at least one processor and at least one memory in communication with the processor, said memory tangibly storing instructions that, when executed by the processor, are configured to at least:
train the computational model to predict T cell-antigen specificity using a sparse data set of known T cell-antigen interactions;
to generate an optimized energy model of amino acid interactions that determine T cell specificity against antigens; and
to predict via the optimized energy model and binding specificity of an antigenic peptide toward a specific T cell receptor based on the respective peptide sequences thereof.

17. The system of claim 16, wherein the electronic device further comprises at least one network connection.

18. The system of claim 16, wherein the sparse data set comprises a plurality of human MHC-I allele variant peptide sequences and protein crystal structures thereof.

19. The system of claim 16, wherein the computational model is configured to predict which T cells recognize an unknown antigen or which unknown T cells recognize a given antigen.


SEQUENCE LISTING (62 sequences)

SEQ ID NO: 1 - protein, 10 aa, synthetic construct
ELAGIGILTV

SEQ ID NO: 2 - protein, 9 aa, synthetic construct
LLFVYPVYV

SEQ ID NO: 3 - protein, 10 aa, synthetic construct
KLVALGINAV

SEQ ID NO: 4 - protein, 10 aa, Synthetic construct
ELAGIGILTV

SEQ ID NO: 5 - protein, 9 aa, synthetic construct
GILGFVFTL

SEQ ID NO: 6 - protein, 9 aa, synthetic construct
ILKEPVHGV

SEQ ID NO: 7 - protein, 9 aa, synthetic construct
CVNGSCFTV

SEQ ID NO: 8 - protein, 10 aa, synthetic construct
MFRHTSMISK

SEQ ID NO: 9 - protein, 10 aa, synthetic construct
KEKEGEAKGA

SEQ ID NO: 10 - protein, 10 aa, synthetic construct
VCIDNLSWDK

SEQ ID NO: 11 - protein, 10 aa, synthetic construct
ALWGPDPAAA

SEQ ID NO: 12 - protein, 10 aa, synthetic construct
KARTTSMIGK

SEQ ID NO: 13 - protein, 10 aa, synthetic construct
AEKEIEATGF

SEQ ID NO: 14 - protein, 10 aa, synthetic construct
EEIDALSIDA

SEQ ID NO: 15 - protein, 9 aa, synthetic construct
GILGFVFTO

SEQ ID NO: 16 - protein, 10 aa, synthetic construct
IARGTSMIGE

SEQ ID NO: 17 - protein, 10 aa, synthetic construct
GEIEGEATLT

SEQ ID NO: 18 - protein, 10 aa, synthetic construct
AEIDALSIDG

SEQ ID NO: 19 - protein, 9 aa, synthetic construct
NLVPMVATV

SEQ ID NO: 20 - protein, 8 aa, synthetic construct
LLFGPVYV

SEQ ID NO: 21 - protein, 9 aa, synthetic construct
AVNFGGKLI

SEQ ID NO: 22 - protein, 11 aa, synthetic construct
AMRGDSSYKLI

SEQ ID NO: 23 - protein, 9 aa, synthetic construct
ARNTGNQFY

SEQ ID NO: 24 - protein, 11 aa, synthetic construct
AVTTDSWGKLQ

SEQ ID NO: 25 - protein, 11 aa, synthetic construct
ASSLSFGTEAF

SEQ ID NO: 26 - protein, 14 aa, synthetic construct
ASSLWEKLAKNIQY

SEQ ID NO: 27 - protein, 13 aa, synthetic construct
ASSPVTGGIYGYT

SEQ ID NO: 28 - protein, 14 aa, synthetic construct
ASRPGLAGGRPEQY

SEQ ID NO: 29 - protein, 9 aa, synthetic construct
NLVPMVATV

SEQ ID NO: 30 - protein, 9 aa, synthetic construct
NLVAMVATV

SEQ ID NO: 31 - protein, 9 aa, synthetic construct
GILEFVFTL

SEQ ID NO: 32 - protein, 10 aa, synthetic construct
AAWGPDPAAA

SEQ ID NO: 33 - protein, 9 aa, synthetic construct
NLVGMVATV

SEQ ID NO: 34 - protein, 10 aa, synthetic construct
EAAGIGILTV

SEQ ID NO: 35 - protein, 9 aa, synthetic construct
AAGIGILTV

SEQ ID NO: 36 - protein, 10 aa, synthetic construct
ELAGIAILTV

SEQ ID NO: 37 - protein, 10 aa, synthetic construct
ELAAIGILTV

SEQ ID NO: 38 - protein, 9 aa, synthetic construct
LLWNGPMAV

SEQ ID NO: 39 - protein, 10 aa, synthetic construct
KLVALGINAV

SEQ ID NO: 40 - protein, 9 aa, synthetic construct
GLCTLVAML

SEQ ID NO: 41 - protein, 9 aa, synthetic construct
CINGVCWTV

SEQ ID NO: 42 - protein, 9 aa, synthetic construct
ATDALMTGF

SEQ ID NO: 43 - protein, 9 aa, synthetic construct
GPRLGVRAT

SEQ ID NO: 44 - protein, 8 aa, synthetic construct
HPVTKYIM

SEQ ID NO: 45 - protein, 9 aa, synthetic construct
KSKRTPMGF

SEQ ID NO: 46 - protein, 9 aa, synthetic construct
LTDEMIAQY

SEQ ID NO: 47 - protein, 10 aa, synthetic construct
NQKLIANDQF

SEQ ID NO: 48 - protein, 9 aa, synthetic construct
NYNYLYRLF

SEQ ID NO: 49 - protein, 9 aa, synthetic construct
RAQAPPPSW

SEQ ID NO: 50 - protein, 9 aa, synthetic construct
SPRWYFYYL

SEQ ID NO: 51 - protein, 10 aa, synthetic construct
TPRVTGGGAM

SEQ ID NO: 52 - protein, 9 aa, synthetic construct
TDPSFLGRY

SEQ ID NO: 53 - protein, 9 aa, synthetic construct
YLQPRTFLL

SEQ ID NO: 54 - protein, 9 aa, synthetic construct
NLVPMVATV

SEQ ID NO: 55 - protein, 12 aa, synthetic construct
ASGTGRSSYEQY

SEQ ID NO: 56 - protein, 11 aa, synthetic construct
AENGNQGGKLI

SEQ ID NO: 57 - protein, 9 aa, synthetic construct
AGGSQGNLI

SEQ ID NO: 58 - protein, 10 aa, synthetic construct
AVRDVNARLM

SEQ ID NO: 59 - protein, 14 aa, synthetic construct
ASSLAPGATNEKLF

SEQ ID NO: 60 - protein, 10 aa, synthetic construct
ASSPGLGYGY

SEQ ID NO: 61 - protein, 13 aa, synthetic construct
ASSLGAISTDTQY

SEQ ID NO: 62 - protein, 11 aa, synthetic construct
ASSIRSGYEQY